(19) United States
(12) Patent Application Publication
(10) Pub. No.: US 2023/0346864 A1
(43) Pub. Date: Nov. 2, 2023

(54) Compositions and Methods for Joint Health

(71) Applicant: Innophos, LLC, Cranbury, NJ (US)

(72) Inventors: Jatinder Rana, Grand Rapids, MI (US); Nylen Simmons, Helmetta, NJ (US)

(73) Assignee: Innophos, LLC, Cranbury, NJ (US)

(21) Appl. No.: 18/218,839

(22) Filed: Jul. 6, 2023

Related U.S. Application Data
(62) Division of application No. 17/227,946, filed on Apr. 12, 2021.
(60) Provisional application No. 63/166,458, filed on Mar. 26, 2021.
(60) Provisional application No. 63/021,406, filed on May 7, 2020.

(51) Int. Cl. A61K 36/22; A23L 33/105; A61P 19/02

(52) U.S. Cl. CPC A61K 36/22; A23L 33/105; A61P 19/02; A61K 2236/333

(57) ABSTRACT

Compositions for and methods of maintaining bone structure, cartilage structure or both, minimizing bone reabsorption, preventing cartilage degradation, increasing bone density, promoting healthy joints by protecting cartilage integrity, diminishing the action of enzymes that affect bone health, cartilage health, or both, improving joint movement or function, alleviating joint pain, alleviating joint discomfort, alleviating joint pain and discomfort, alleviating joint stiffness, improving joint range of motion or flexibility, promote mobility, or the like, wherein the composition comprises a therapeutically effective amount of a botanical extract of the testa of Anacardium occidentale L, wherein the botanical extract is enriched for total catechin content.

### CROSS REFERENCE TO RELATED APPLICATIONS

The present application is a divisional application of U.S. patent application Ser. No. 17/227,946, filed 4 Apr. 2021, which claims the benefit of U.S. Application No. 63/021,406 filed 7 May 2020 and U.S. Application No. 63/166,458 filed 26 Mar. 2021, the disclosures of which are incorporated herein by reference.

### BACKGROUND OF THE INVENTION

Field of the Invention. The present invention generally relates to a botanical extract or compositions thereof comprising the botanical extract that can modulate joint inflammation, joint pain, joint stiffness, cartilage degradation, or improving mobility, range of motion, flexibility, joint physical function, or any combinations thereof. The present invention further can optionally be used in combination with other joint management agents, such as calcium, magnesium, zinc, boron, Vitamin D, Vitamin K, glucosamine and/or chondroitin compounds, non-steroidal anti-inflammatory agents/analgesics, COX/LOX inhibiting agents, glucosamine compounds, neuropathic pain relief agents, or the like.

Rheumatoid arthritis (‘RA’) is one of the most prevalent chronic autoimmune diseases. Its early stages involve local swelling and stiffness in synovial joints before advancing to a chronic multisystem disease. Increases in both the cellularity of synovial tissue and joint damage due to inflammatory reactions are the pathological features of RA. Key inflammatory cascades in RA involve systemic overproduction and expression of pro-inflammatory cytokines such as interleukin-6 (‘IL-6’) and tumor necrosis factor-α (‘TNF-α’), accelerating bone/joint complications. Synovial inflammation in RA spreads systemically and transforms silently into chronic inflammation manifested by increased cytokine release (e.g., IL-1β, IL-6, and IL-18) and abnormally high levels of acute reactive proteins (‘ARPs’) such as C-reactive protein (‘CRP’), leading to continuous inflammation and joint damage.

Accordingly, the pathology of RA is complex, and etiology underlying RA remains unknown. Destructive changes in cartilage and bone, and bony outgrowths restricting mobility of the joint occur. Arthritis can cause severe disability, and ultimately affects a person's ability to carry out everyday tasks, restrict the quality of life, and causes premature death. Any part of the body can become inflamed or painful from arthritis. It is one of the most common inflammatory disorders, affecting approximately 0.5-1.0% of the global adult population, with females being affected three times more than males.

Although currently available treatments have improved efficiency, the use of non-steroidal anti-inflammatory drugs (NSAIDs), such as indomethacin; disease-modifying anti-rheumatoid drugs (DMARDs), such as methotrexate, sulfasalazine, leflunomide, and hydroxychloroquine; and corticosteroids, such as prednisolone and methylprednisolone, is associated with several adverse reactions. Hence, patients with musculoskeletal disorders have sought alternative methods for symptomatic relief.

Arachidonic acid and its metabolites are important mediators of inflammation. Arachidonic acid (‘AA’) is a component of membrane phospholipids where the rate-limiting step in the formation of its metabolites depends on its release from the cell membrane phospholipid pool mediated through activation of phospholipases. Phospholipase A2 (‘PLA2’) activity is increased in arthritis, and cytokines including TNF-α and IL-1 have been reported to stimulate the activity of PLA2. After its release, AA can be metabolized by one of two pathways—by cyclooxygenase (‘COX’) to yield eicosanoids such as prostaglandins (‘PGE2’), prostacyclins, and thromboxanes, or it can be metabolized by 5-lipoxygenase (‘5-LOX’) to result in the production of leukotrienes and lipoxins. These eicosanoids serve as intracellular messengers and play significant roles in the regulation of signal transduction in pain and inflammatory responses. An illustration of the arachidonic acid metabolism pathway is provided in FIG. 1.

Cyclooxygenase—a prostanoid synthase also known as prostaglandin-endoperoxide synthase (PTGS, EC 1.14.99.1)—is an enzyme that is responsible for the formation of important biological mediators called prostanoids, including prostaglandins, prostacyclin and thromboxane. COX is the central enzyme in the biosynthetic pathway to prostanoids from arachidonic acid. There are two known isoenzymes—COX-1 and COX-2. COX-1 represents the constitutive isoform responsible for production of prostaglandins involved in physiological functions such as protection of the gastric mucosa and maintenance of renal perfusion. COX-2 is not expressed under normal conditions in most cells, but elevated levels are found during inflammation. COX-2 is the dominant isozyme in inflamed tissues, where its induction can be facilitated by several pro-inflammatory cytokines, including interleukin-1 (‘IL-1’) and tumor necrosis factor (TNF-α′). Pharmacological inhibition of COX by non-steroidal anti-inflammatory drugs (NSAID) can provide relief from the symptoms of inflammation and pain.

Therefore, to prevent the unwanted side effects, it seems practical to inhibit COX-2 selectively for its analgesic and anti-inflammatory effects without affecting important physiological processes controlled by the prostaglandins formed by COX-1. Still, there are reports that associate the synergistic effect of COX-2 as a constitutive isoenzyme in maintaining renal blood flow and the glomerular filtration rate suggesting its selective inhibition may lead to some adverse effects. These effects were experienced by subjects in clinical trials wherein selective COX-2 inhibitors (e.g., celecoxib and rofecoxib) provided similar efficacy to that of traditional NSAIDs in osteoarthritis and rheumatoid arthritis pain with better gastric tolerability and equivalent to NSAIDs in renal side effects. Therefore, it is reasonable to assume and have a compound strong enough to cause inhibition of these isoenzymes yet moderate enough to avoid the unnecessary adverse consequences, as opposed to a complete selective inhibition of either of the enzymes.

Increased expression of COX-2, and hence synthesis of its product PGE2, has also been found to be strongly associated with the induction of MMP-9, which is a key player in cancer, cardiovascular disease, and inflammation. Therefore, inhibition of COX-2 enzyme may result in regulation of MMP-9 expression and activity that may modulate invasion and migration of cancer cells, prevent or delay the progression of atherosclerosis and stabilize plaques, regulate macrophage proteinase expression, prevent chronic periodontitis and gingivitis, and control remodeling of liver disease, among others.

The other segment of the Arachidonic acid (‘AA’) metabolism pathway is through the 5-lipoxygenase (‘5-LOX’) pathway, where leukotrienes (LTB4, LTC4, LTD4, and LTE4) derived from LTA4 are the end bioactive metabolites. It is known that lipoxygenase pathways are important in the rheumatoid arthritis (‘RA’) inflammatory process, and that synovial fluid from RA patients contains high amounts of leukotrienes. For example, 5-LOX is present in RA and OA synovium, with 5-LOX being mostly expressed in lining and sublining macrophages, neutrophils, and mast cells. The other component of this pathway—LTB4, a downstream product of 5-LOX—is a potent proinflammatory chemotactic agent and has been implicated as an important mediator of joint inflammation in RA. There are higher levels of LTB4 in the serum of RA patients that patients with inactive arthritis or normal subjects. While a specific inhibitor of the 5-LOX enzyme—PF-4191834 from Pfizer—is found to decrease arthritis-associate pain and inflammation in rat model, single therapeutic modality for 5-LOX modulators seems insufficient.

Preferably, anti-inflammatory products encompass inhibition of both main metabolic pathways of Arachidonic acid (‘AA’) metabolism, possessing a wide range of anti-inflammatory activities while also having a better safety profile.

Another mediator of inflammation which acts as cytokine and is secreted by immune cells are High Mobility Group Box 1 proteins (‘HMGB1’), also known as high-mobility group protein 1 (‘HMG-1’) and amphoterin. HMGB1 is a protein that in humans is encoded by the HMGB1 gene. Like the histones, HMGB1 is among the most important chromatin proteins. HMGB1 is a 30 kDa nuclear and cytosolic protein and is a self-derived immune activator that has multiple functions in the regulation of immunity and inflammation.

HMGB1 can be released actively by innate immune cells such as macrophages, monocytes, and dendritic cells at the time of inflammation and injury. For example, macrophages and monocytes actively release HMGB1 in a time- and dose-dependent manner in response to stimulation with exogenous bacterial endotoxin (e.g., lipopolysaccharide, or LPS), or endogenous pro-inflammatory cytokines such as tumor necrosis factor (‘TNF-α’), Interleukin-1 beta (‘1L-1β’), and Interferon gamma (‘IFN-γ’).

HMGB1 can also be released passively by necrotic or damaged cells and can induce an inflammatory response by communicating the insult to the neighboring immune cells, allowing the innate immune cells to both respond to injury and to further induce inflammation. HMGB1 proteins trigger intracellular signaling through receptor for advanced glycosylation end products (‘RAGE’) and/or Toll-like receptors (TLR-2/4), which in turn activate various signaling pathways as mitogen-activated protein kinase (‘MARK’) pathways and subsequent nuclear factor kappa-light-chain-enhancer of activated B cells (‘NF-κB’) mediating inflammation, leading to the expression of various leukocyte adhesion molecules, pro-inflammatory cytokines, and chemokines.

HMGB1 plays significant roles in inflammatory activity and is involved in a wide range of immune responses. HMGB1 induces maturation and migration of dendritic cells (‘DCs’), as well as the activation of these cells and monocytes to produce pro-inflammatory cytokines such as TNF-α, IL-1β, IL-6, and macrophage inflammatory protein 1 (‘MIP-1’). HMGB1 also serves as a chemotactic factor for monocytes, macrophages, neutrophils, and DCs to sustain inflammation and elicit innate immune response.

HMGB1 is considered a lead example of a danger signal that originates from the damaged self instead of from invading pathogens. HMGB1 mediates activation of innate receptors resulting in the amplification of inflammatory responses through the release of cytokines, which in turn induce the release of additional HMGB1, further promoting the induction of these mediators. While pro-inflammatory cytokines such as TNF-α, IL-10, and IFN-γ are known to mediate the early phases of inflammation, HMGB1 is considered as the late phase dictator in sepsis and tissue injury.

Targeting HMGB1 may be a pragmatic approach for therapeutic interventions in inflammatory diseases as it has been identified as a crucial mediator in the pathogenesis of many diseases, including sepsis, arthritis, cancer, and diabetes. For example, the level of HMGB1 has been found to be elevated in (1) synovial fluid of patients with rheumatoid arthritis, (2) septic patients who did not survive compared to those who did survive, (3) invasion and metastasis of solid tumors, and (4) diabetes and its complications.

As a consequence, many pharmacologic agents have been studied for their potential to inhibit release of HMGB1 or HMGB1 activity (see, FIG. 2). These include traditional herbal medicines such as aqueous extracts of dong guai or dang gui (“female ginseng”—Angelica sinensis), Green tea (Camellia sisensis), and Danshen (“red sage” or “Chinese sage”—Saliva miltorrhiza), which have been found to inhibit endotoxin-induced HMGB1 release, as well as protect animals against experimental sepsis. Scientific research has demonstrated that these herbal extracts have strong anti-inflammatory and anti-arthritic effects. A wide range of phytochemicals including polysaccharides, phenolic acids, phenylpropanoid ester, triterpene glycosides, phthalide, flavonoids, triterpenoid saponin, diterpene and triterpene have been isolated and demonstrated to be responsible for the biological effects of the herbs.

Accordingly, phytomedicine plays an important role in the management of most of these diseases, with plants being a potential source of natural antioxidants. Studies have shown that the consumption of polyphenolic compounds found in tea, herbs, fruits, and vegetables is associated with low risk of these diseases. Consequently, there is a growing research interest in plants that exhibit anti-inflammatory activity and health-promoting phytoconstituents as potential therapeutic agents. Medicinal plants can provide a safe, cost-effective, ecological alternative to chemical antioxidants, which can be toxic on prolonged exposure.

The cashew tree (Anacardium occidentale Linn) is originally from the Amazon, and has subsequently been transplanted to India, Eastern Africa, and other countries for cultivation. The tree produces a very peculiar apple or fruit in the form of a swollen peduncle. Externally at the end of this peduncle the cashew nut grows in its own grey colored kidney-shaped hard shell. This shell has a soft leathery outer skin and a thin hard inner skin referred to as the husk or testa, which surrounds the kernel. Between these two skins is a honeycomb structure containing the cashew nutshell liquid. This liquid comprises anacardic acid, cardanol, and cardol, among other ingredients. Anacardic acid is a salicylic acid, while cardanol and cardol are substituted phenols.

The various parts of the fruit have been studied for their uses. In addition to being an edible food, the juice from the cashew apple is used in beverages, while the fruit extract has shown benefit in weight management. Cashew nut shell liquid has been extracted for various industrial and agricultural applications, include friction linings, paints, laminating resins, rubber compounding resins, cashew cements, polyurethane based polymers, surfactants, epoxy resins, foundry chemicals, chemical intermediates, insecticides, and fungicides. Cashew testa has been used in tanning materials.

As noted above, there is a need for effective, nontoxic, natural compounds with anti-inflammatory activity. More specifically, there is a need for effective, nontoxic, natural compounds having joint inflammation, joint pain, joint stiffness, cartilage degradation modulatory efficacy. The present invention provides one such solution.

### BRIEF SUMMARY OF THE INVENTION

In brief, the present disclosure is directed towards standardized botanical extracts and compositions containing those extracts that are useful for joint health management, and to related methods of improving joint health.

More specifically, provided herein is a botanical extract composition comprising catechins, wherein the extract has been standardized to a total catechin content of about 15.0 wt % or greater, based on total weight of the extract. The botanical extract composition exhibits promoted joint health due to its anti-inflammatory activity and comprises at least an extract from the genus Anacardium. Preferably the botanical extract is at least an extract from Anacardium occidentale L. More preferably, the botanical extract is from at least the testa of the fruit of Anacardium occidentale L.

In one embodiment, the present invention is directed towards an extract of the testa of the fruit of Anacardium occidentale L. comprising about 15.0 wt % or greater total catechins, based on total weight of the extract.

In a further embodiment, the present invention provides a composition for reducing joint stiffness and discomfort in a mammal in need thereof comprising a therapeutically effective amount of a botanical extract of the testa of Anacardium occidentale L, wherein the botanical extract is enriched for total catechin content. The botanical extract can be further enriched for total polyphenols.

In one embodiment, the therapeutically effective amount of the botanical extract in the composition for reducing joint stiffness and discomfort in a mammal in need thereof can be in at least an amount of about 500.0 mg/kg or greater, based on human equivalent dosing. In a further embodiment, the therapeutically effective amount of the botanical extract in the composition is an amount of about 500.0 mg/kg to about 2000.0 mg/kg, based on human equivalent dosing. In an even further embodiment, the therapeutically effective amount of the botanical extract in the composition is an amount of about 1000.0 mg/kg to about 2000.0 mg/kg, based on human equivalent dosing.

In one embodiment, the botanical extract in the composition for reducing joint stiffness and discomfort in a mammal in need thereof is standardized to a total catechin content of at least about 15.00% by weight, based on total weight of the extract.

In one embodiment, the composition for reducing joint stiffness and discomfort in a mammal in need thereof alleviates cyclooxygenase and 5-lipoxygenase mediated inflammation in the mammal having joint stiffness and discomfort.

In one embodiment, the composition for reducing joint stiffness and discomfort in a mammal in need thereof further comprises a pharmaceutical carrier.

The composition for reducing joint stiffness and discomfort in a mammal in need thereof can be a dietary supplement.

In another aspect, the present invention provides a composition for improving cartilage rebuild or renewal function in a mammal in need thereof comprising a therapeutically effective amount of a botanical extract of the testa of Anacardium occidentale L, wherein the botanical extract is enriched for total catechin content.

The therapeutically effective amount of the botanical extract in the composition for improving cartilage rebuild or renewal function in a mammal in need thereof can be at least an amount of about 500.0 mg/kg or greater, based on human equivalent dosing. Preferably, the therapeutically effective amount of the botanical extract in the composition is an amount of about 500.0 mg/kg to about 2000.0 mg/kg, based on human equivalent dosing. More preferably, the therapeutically effective amount of the botanical extract in the composition is an amount of about 1000.0 mg/kg to about 2000.0 mg/kg, based on human equivalent dosing.

In a further embodiment, the botanical extract in the composition for improving cartilage rebuild or renewal function in a mammal in need thereof is standardized to a total catechin content of at least about 15.00% by weight, based on total weight of the extract.

In one embodiment, the composition for improving cartilage rebuild or renewal function in a mammal in need thereof alleviates cyclooxygenase and 5-lipoxygenase mediated inflammation in the mammal needing cartilage rebuild or renewal function.

The composition for improving cartilage rebuild or renewal function in a mammal in need thereof can further comprise a pharmaceutically acceptable carrier.

Further, the composition for improving cartilage rebuild or renewal function in a mammal in need thereof can be a dietary supplement.

In another aspect, a method for reducing joint stiffness and discomfort in a mammal in need thereof is provided comprising administering a therapeutically effective amount of a composition comprising a botanical extract of the testa of Anacardium occidentale L, wherein the botanical extract is enriched for total catechin content.

In one aspect, the botanical extract in method for reducing joint stiffness and discomfort in a mammal in need thereof is standardized to a total catechin content of at least about 15.00% by weight, based on total weight of the extract. The botanical extract can be further enriched for total polyphenols.

In one aspect, the method for reducing joint stiffness and discomfort in a mammal in need thereof alleviates cyclooxygenase and 5-lipoxygenase mediated inflammation in the mammal having joint stiffness and discomfort.

In another embodiment, a method for improving cartilage rebuild or renewal function in a mammal in need thereof is provided comprising administering a therapeutically effective amount of a composition comprising a botanical extract of the testa of Anacardium occidentale L, wherein the botanical extract is enriched for total catechin content. The botanical extract is further enriched for total polyphenols.

The botanical extract in the method for improving cartilage rebuild or renewal function in a mammal in need thereof can be standardized to a total catechin content of at least about 15.00% by weight, based on total weight of the extract.

In one aspect, the method for improving cartilage rebuild or renewal function in a mammal in need thereof alleviates cyclooxygenase and 5-lipoxygenase mediated inflammation in the mammal in need of cartilage rebuild or renewal function.

Compositions containing the botanical extract of the testa of Anacardium occidentale L. can further comprise a pharmaceutically acceptable carrier. Non-limiting examples of such compositions include dietary supplements and topical compositions.

### BRIEF DESCRIPTION OF THE SEVERAL VIEWS OF THE DRAWINGS

FIGS. 1A and 1B is a general illustration of the Arachiconic acid metabolism pathway.

FIG. 2 is a general illustration of HMGB1-mediated pro-inflammatory responses at various sites.

FIG. 3 is an HPLC chromatogram of cashew testa extract at 275 nm wavelength over a retention time of from 0 minutes (start) to 20 minutes.

FIG. 4 is LC/MS and LC/PDA (wavelengths of 280 and 350 nm) chromatograms of cashew testa extract.

FIG. 5 is a graph illustrating percentage COX-1 inhibition using cashew testa extract at various concentrations.

FIG. 6 is a graph illustrating percentage COX-2 inhibition using cashew testa extract at various concentrations.

FIG. 7 is a graph illustrating percentage 5-LOX inhibition using cashew testa extract at various concentrations.

FIG. 8 is a bar graph illustrating the detection of HMGB1 (% release) in macrophage cell culture supernatant at room atmosphere (21% 02) (‘RA’), 95% 02 (‘02’) without cashew testa extract, DMSO (‘Vehicle’), positive control sodium salicylate (‘SS 2 μM’), and 95% 02 with cashew testa extract (‘CT’).

FIG. 9 is a process diagram illustrating the cartilage-induced arthritis (‘CIA’) experimental design utilized.

FIG. 10 is a graph illustrating the changes in arthritis severity index from day 9 to day 21 for each study group.

FIG. 11 is a bar chart illustrating the area under arthritis severity score curve (‘AUC’) for each study group.

FIG. 12 is a graph illustrating the changes in paw thickness from priming to day 21 for each study group.

FIG. 13 is bar chart illustrating the area under the curve for rats' paw edema for each study group in the CIA model.

FIG. 14 is a graph illustrating the changes in rats' ankle diameter as a measure of arthritis severity from priming to day 21 for each study group in the CIA model.

FIG. 15 is a bar chart illustrating the area under the curve for rats' ankle diameter for each study group in the CIA model.

FIG. 16 is a graph illustrating the pain sensitivity response of the rats in each study group from priming to day 21 in the CIA model.

FIG. 17 is a bar chart illustrating the percentage change in compression threshold measured as a percentage of pain sensitivity improvement from priming to day 21 for each study group in the CIA model.

FIG. 18 is a bar chart illustrating the urine CTX-II raw data without normalization.

FIG. 19 is a bar chart illustrating the urine CTX-II normalized to total protein.

FIG. 20 is a bar chart illustrating the urine CTX-II normalized to creatinine concentration in urine.

FIG. 21 is a bar chart illustrating the serum IL-1β for each study group in the CIA model for 3 weeks post model induction.

FIG. 22 is a bar chart illustrating the serum TNF-α for each study group in the CIA model for 3 weeks post model induction.

FIG. 23 is a bar chart illustrating the serum PIIANP for each study group in the CIA model for 3 weeks post model induction.

FIG. 24 is a bar chart illustrating the serum MMP-13 for each study group in the CIA model for 3 weeks post model induction.

FIG. 25 are four bar charts illustrating the histopathology findings of CIA rats' ankle joint for each study group for (A) cartilage destruction, (B) bone erosion, (C) inflammation, and (D) matrix integrity/GAG loss.

FIG. 26 are hematoxylin and eosin and safranin 0-fast green stains of ankle joint sections for each study group.

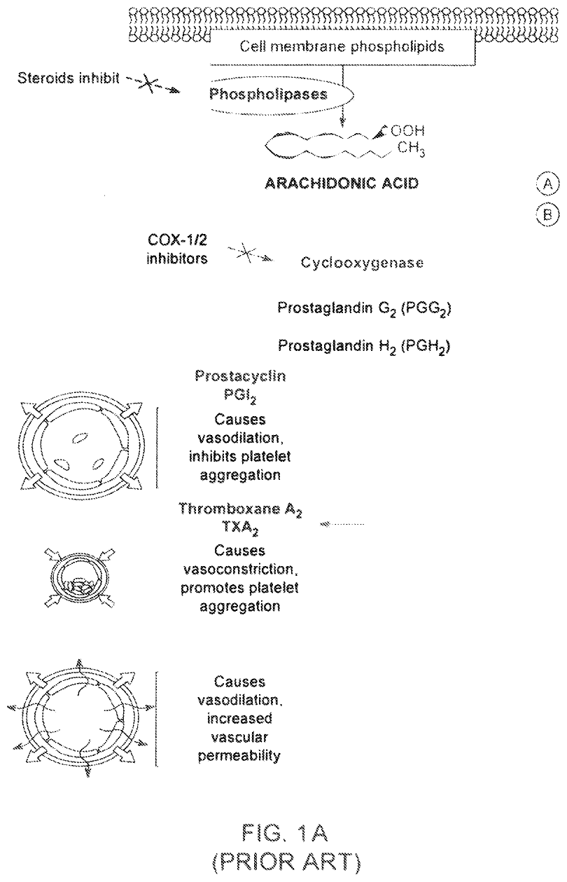

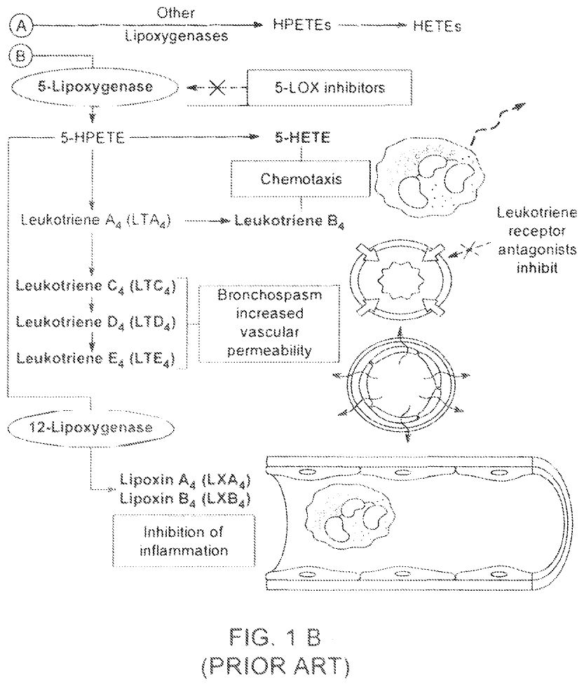

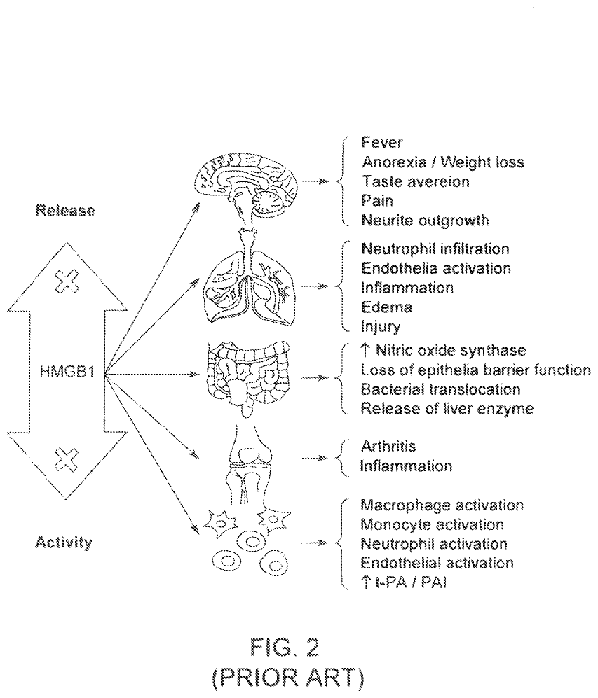

### DETAILED DESCRIPTION OF THE INVENTION

The present invention is based on the surprising discovery that the testa of the cashew (Anacardium occidentale Linn) is substantially high in certain flavonoids. In particularly, it has been discovered that the extract of cashew testa comprises catechin and epicatechin as major components, as well as procyanidins. Data noted herein demonstrates that cashew testa extract may have anti-inflammatory applications.

Other aspects of the present invention relate to methods of using compositions of this disclosure, such as for maintaining bone structure, cartilage structure or both, minimizing bone reabsorption, preventing cartilage degradation, increasing bone density, promoting healthy joints by protecting cartilage integrity, diminishing the action of enzymes that affect bone health, cartilage health, or both, improving joint movement or function, alleviating joint pain, alleviating joint discomfort, alleviating joint pain and discomfort, alleviating joint stiffness, improving joint range of motion or flexibility, promote mobility, or the like.

In the following description, certain specific details are set forth in order to provide a thorough understanding of various embodiments of this disclosure. However, one skilled in the art will understand that the invention may be practiced without these details.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “consisting essentially of” mean±20% of the indicated range, value, or structure, unless otherwise indicated. The terms “a” and “an” as used herein refer to “one or more” of the enumerated components. The use of the alternative (e.g., “or”) should be understood to mean either one, both, or any combination thereof of the alternatives. Unless the context requires otherwise, throughout the present specification and claims, the word “comprise” and variations thereof, such as, “comprises” and “comprising,” as well as synonymous terms like “include” and “have” and variants thereof, are to be construed in an open, inclusive sense; that is, as “including, but not limited to”.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

For the present application, the term “composition” refers to a product that treats, improves, promotes, increases, manages, controls, maintains, optimizes, modifies, reduces, inhibits, or prevents a particular condition associated with a natural state, biological process or disease or disorder. For example, a composition improves the inhibition of oxidation and/or reduces inflammation, and the like in a subject. The term composition includes, but is not limited to, pharmaceutical (i.e., drug), over-the counter (OTC), cosmetic, food, food ingredient or dietary supplement compositions that include an effective amount of an extract, at least one component thereof, or a mixture thereof. Exemplary compositions include cream, cosmetic lotion, pack or powder, or as an emulsion, lotion, liniment foam, tablets, plasters, granules, or ointment. Compositions can also include beverages, for example, beverages infused with an effective amount of an extract, or a tea satchel containing an effective amount of an extract. Non-limiting examples of food compositions containing an effective amount of an extract include baked goods, protein powders, meat products, dairy products, and confectionary.

Further, a “pharmaceutical composition” or “nutraceutical composition” as used herein refers to a formulation of a botanical extract of this disclosure and a medium generally accepted in the art for the delivery of the biologically active extract to mammals, e.g., humans. For example, a pharmaceutical composition of the present disclosure may be formulated or used as a stand-alone composition, or as a component in a prescription drug, an over-the-counter (OTC) medicine, a botanical drug, an herbal medicine, a homeopathic agent, functional foods, or any other form of health care product reviewed and approved by a government agency. Exemplary nutraceutical compositions of the present disclosure may be formulated or used as a stand-alone composition, or as a nutritional or bioactive component in food, a novel food, a functional food, a beverage, a bar, a food flavor, a food additive, a medical food, a dietary supplement, or an herbal product. A medium generally accepted in the art includes all pharmaceutically or nutraceutically acceptable carriers, diluents, or excipients therefor.

As used herein, the term “extract” or “botanical extract” refers to a solid, viscid, or liquid substance or preparation that includes one or more active ingredients of a substance of at least the plant genus Anacardium (e.g., Anacardium humile, Anacardium othonianum, Anacardium giganteum, Anacardium nanum, Anacardium negrense, and/or Anacardium occidentale), preferably Anacardium occidentale L. Preferably, the active ingredient is derived from the extract of the testa of the cashew. The extract is prepared using a solvent such as water, lower alcohols of 1 to 4 carbon atoms (e.g., methanol, ethanol, butanol, etc.), ethylene, acetone, hexane, ether, chloroform, ethyl acetate, butyl acetate, dichloromethane, N,N-dimethylformamide (‘DMF’), dimethyl sulfoxide (‘DMSO’), 1,3-butylene glycol, propylene glycol, and combinations thereof, but also a fraction of the crude extract in such a solvent. So long as it assures the extraction and preservation of the active ingredient(s), any extraction method may be employed.

As used herein, “enriched for” refers to a plant extract or other preparation having at least a two-fold up increase in the amount of or activity of one or more active compounds as compared to the amount or activity of the one or more active compounds found in the weight of the raw plant material or other source before extraction or other preparation. In certain embodiments, the weight of the plant material or other source before extraction or other preparation may be dry weight, wet weight, or a combination thereof.

As used herein, the term “effective amount” or “therapeutically effective amount” of a pure compound, composition, extract, extract mixture, component of the extract, and/or active agent or ingredient, or a combination thereof refers to an amount effective at dosages and for periods of time sufficient to achieve a desired result. More specifically, “effective amount” or “therapeutically effective amount” refers to that amount of an extract or composition containing the extract of this disclosure that, when administered to a mammal, such as a human, is sufficient to effect treatment, including any one or more of: (1) treating or preventing loss of bone and cartilage in a mammal; (2) promoting bone and cartilage health; (3) suppressing loss of bone and cartilage in a mammal; (4) increasing bone density in a mammal; (5) treating or preventing osteoporosis in a mammal; (6) modifying inflammation of bone and cartilage in a mammal; (7) protecting bone and cartilage integrity; and (8) reducing joint stiffness and discomfort. The amount of a compound or composition of this disclosure that constitutes a “therapeutically effective amount” will vary depending on the amount of the major active ingredient, the condition being treated and its severity, the manner of administration, the duration of treatment, or the body weight and age of a subject to be treated but can be determined by one of ordinary skill in the art having regard to his own knowledge and to this disclosure.

The term “pharmaceutically acceptable” means those drugs, medicaments, extracts or inert ingredients, which are suitable for use in contact with the tissues of humans and lower animals without undue toxicity, incompatibility, instability, irritation, and the like, commensurate with a reasonable benefit/risk ratio.

The terms “administer”, “administered”, “administers”, and “administering” are defined as providing a composition to a subject via a route known in the art, including but not limited to intravenous, intra-arterial, oral, parenteral, buccal, topical, transdermal, rectal, intramuscular, subcutaneous, intraosseous, transmucosal, or intraperitoneal routes of administration. In preferred embodiments, oral routes of administering a composition are suitable.

As used herein, the term “subject” or “individual” includes mammals to which a composition may be administered. Non-limiting examples of “mammals” include humans, non-human primates, canines, felines, equines, bovines, rodents (including transgenic and non-transgenic mice) or the like. In some embodiments, the subject is a non-human mammal, and in some embodiments, the subject is human.

As used herein, the term “carrier” refers to a composition that aids in maintaining one or more plant extracts in a soluble and homogeneous state in a form suitable for administration, which is nontoxic, and which does not interact with other components in a deleterious manner.

“Supplements” as used herein refers to a product that improves, promotes, supports, increases, regulates, manages, controls, maintains, optimizes, modifies, reduces, inhibits, or prevents a particular condition, structure or function associated with a natural state or biological process (i.e., are not used to diagnose, treat, mitigate, cure, or prevent disease). In certain embodiments, a supplement is a dietary supplement. For example, with regard to bone and cartilage health-related conditions, dietary supplements may be used to maintain bone and cartilage integrity, minimize bone reabsorption, minimize cartilage degradation, promote healthy bone and cartilage by protecting bone and cartilage integrity, diminish the action of enzymes that affect bone and cartilage health, improve osteoporosis condition, support bone rebuild, alleviate pain, alleviate discomfort, alleviate stiffness, improve range of motion, improve flexibility, promote mobility, or the like. In certain embodiments, dietary supplements are a special category of diet, food, or both, and are not a drug.

Unless indicated otherwise, all proportions and percentages recited throughout this disclosure are by weight.

In certain embodiments, compounds and compositions (e.g., pharmaceutical, nutraceutical) of the present disclosure may be administered in an amount sufficient to promote bone health; improve bone health; maintain bone health; treat or manage bone disorders; support bone health; support a normal and comfortable range of motion and/or flexibility; improve range of motion and/or flexibility; reduce the action of harmful enzymes that break down bones; alter the action of enzymes that affect bone absorption; improve movement with normal bone function; improve physical mobility; manage and/or maintain physical mobility; alleviate pain and/or stiffness due to bone loss; improve physical function; promote or enhance flexibility and comfortable movement; promote healthy bone function and comfort; relieve bone discomfort; relieve bone discomfort caused by exercise, work, overexertion or any combination thereof; promote healthy bones by protecting cartilage integrity; maintain joint cartilage; support joint cartilage; treat, prevent, or manage cartilage degradation; minimize cartilage degradation; promote joint health or comfort by maintaining synovial fluid for joint lubrication; support joint stability and joint flexibility; revitalize joints and promote mobility; promote flexible joints and strong cartilage; maintain steady blood flow to joints to support enhanced flexibility and/or strength; promote joint comfort and a wide range of motion after exercise, work, overexertion, or any combination thereof; or any other associated indication described herein, and generally with acceptable toxicity to a patient.

The present invention provides a botanical extract that exhibits anti-inflammatory activity and thus promotes joint health. More particularly, the present invention is directed towards a botanical extract of the cashew testa from the genus Anacardium. As shown herein, such botanical extracts have been found to reduce joint stiffness and discomfort and improve joint function. Further, botanical extracts according to the invention provide cartilage protection based on reduction of μCTX-II and protection of joint structure integrity. Botanical extracts according to the invention provide improved cartilage rebuild or renewal. Finally, botanical extracts according to the invention appear to be more effective than glucosamine/chondroitin supplements in improving symptoms of OA, suppressing catabolic pathways, protecting joint structure integrity, and improving cartilage rebuild or renewal function.

As previously stated, useful joint health botanical extracts according to the present invention include botanical extracts from the genus Anacardium. More particularly, the extract is a botanical extract chosen from one or more of the species Anacardium humile, Anacardium othonianum, Anacardium giganteum, Anacardium nanum, Anacardium negrense, and/or Anacardium occidentale. Preferably, the botanical extract is from the species Anacardium occidentale L. In one embodiment, the botanical extract is from the testa of the species Anacardium occidentale L.

Joint health compositions according to the present invention may include one or more compounds that may function as active ingredients. The compound may be a component of the botanical extract. For example, the compound can be a phytochemical present in the plant from which the plant extract is obtained. The compound may be at least partially responsible for exhibiting anti-inflammatory activity. The compound can be any compound capable of promoting joint health. In one embodiment, the compound is chosen from the phytochemicals catechins, epicatechins, and/or procyanidins (e.g., A, B, trimer, tetramer).

Generally, one or more parts of a plant can be used to produce a plant extract including, but not limited to, the root, the stem, the leaf, the flower, the fruit, the seed, and the testa of the seed. In the present invention, at least the testa of the seed is used—alone or with other plant parts—to produce the plant extract. The testa from the Anacardium plant can be commercially obtained from various sources. The extract of the cashew testa can be obtained using any suitable extraction technique.

In this regard, one or more parts of the plant, particularly the testa of the plant, can be collected and milled. Thereafter, the milled material can be extracted using a suitable solvent. The solvent can be removed in a concentration step. For example, the extracted material can be screened or filtered to create a supernatant and a cake. The cake can be pressed to remove a substantial portion of the liquid, which can be added to the supernatant. The cake can then be dehydrated and used as a fiber source. The supernatant can be distilled to remove the solvent or a portion thereof, to form a plant extract liquid concentrate. The removed solvent can be recycled. The concentrate can be dried (e.g., by spray drying) to provide a dried plant extract. This dried plant extract can be assayed and/or standardized as described herein. Preferably, the dried plant extract is derived from Anacardium occidentale, particularly the testa of the plant Anacardium occidentale L.

Suitable solvents for the extraction process include water, alcohol, or mixtures thereof. Exemplary alcoholic solvents include, but are not limited to, C₁-C₇ alcohols (e.g., methanol, ethanol, propanol, isopropanol, and butanol), hydro-alcohols or mixtures of alcohol and water (e.g., hydro-ethanol), polyhydric alcohols (e.g., propylene glycol and butylene glycol), and fatty alcohols. Any of these alcoholic solvents can be used in the form of a mixture. In one embodiment, the plant extract is extracted using ethanol, water, or a combination thereof (e.g., a mixture of about 70% ethanol and about 30% water). In another embodiment, the plant extract is extracted using only water.

In one embodiment, the plant extract can be obtained using an organic solvent extraction technique. In another embodiment, solvent sequential fractionation can be used to obtain the plant extract. Total hydro-ethanolic extraction techniques can also be used to obtain the plant extract. Generally, this is referred to as a lump-sum extraction.

Total ethanol extraction can also be used. This technique uses ethanol as the solvent. This extraction technique can generate a plant extract having fat soluble and/or lipophilic compounds in addition to water soluble compounds.

Another example of an extraction technique that can be used to obtain the plant extract is supercritical fluid carbon dioxide extraction (‘SFE’). In this extraction procedure, the material to be extracted may not be exposed to any organic solvents. Rather, carbon dioxide can be used as the extraction solvent—with or without a modifier—in super-critical conditions (>31.3° C. and >73.8 bar). Those skilled in the art will appreciate that temperature and pressure conditions can be varied to obtain the best yield of extract. This technique can generate an extract of fat soluble and/or lipophilic compounds, similar to a total hexane and ethyl acetate extraction technique.

The plant extract generated in the process can include a broad variety of phytochemicals present in the extracted material. The phytochemicals can be fat soluble or water soluble. Following collection of the extract solution, the solvent can be evaporated, resulting in the extract. The plant extract can be standardized to a specified amount of a particular compound. For example, the plant extract can be standardized to a specified amount of an active ingredient or phytochemical. In one embodiment, the plant extract is standardized to a catechin content of about 15.0 wt % or greater, based on total weight of the extract.

The amount of plant extract present in the joint health composition can depend upon several factors, including the desired level of inflammation inhibition, the inflammation inhibiting level of a particular plant extract or component thereof, and other factors. Preferably, the plant extract is present in an amount of from about 0.005 wt % or greater, for example, from about 0.005 wt % to about 50.00 wt %, based on total weight of the composition.

The joint health composition can include one or more acceptable carriers. The carrier can aid in enabling incorporation of the plant extract into an anti-inflammatory composition having a suitable form for administration to a subject. A wide number of acceptable carriers are known in the art, and the carrier can be any suitable carrier. The carrier is preferable suitable for administration to animals, including humans, and can be able to act as a carrier without substantially affecting the desired activity of the plant extract and/or any active ingredient. The carrier can be chosen based upon the desired administration route and dosage form of the composition.

Suitable dosage forms include liquid and solid forms. In one embodiment, the composition is in the form of a gel, a syrup, a slurry, or a suspension. In another embodiment, the composition is in a liquid dosage form such as a drink shot or a liquid concentrate. In a further embodiment, the composition is present in a solid dosage form, such as a tablet, a pill, a capsule, a dragée, or a powder. When in liquid or solid dosage form, the composition can be in a food delivery form suitable for incorporation into food for delivery. Examples of suitable carriers for use in solid forms (particularly tablet and capsule forms) include, but are not limited to, organic and inorganic inert carrier materials such as gelatin, starch, magnesium stearate, talc, gums, silicon dioxide, stearic acid, cellulose, and the like. The carrier can be substantially inert.

As an example, silicified microcrystalline cellulose can be used as a carrier or binder. Silicified microcrystalline cellulose is a physical mixture of microcrystalline cellulose and colloidal silicon dioxide. One such suitable form of silicified microcrystalline cellulose is ProSolv SMCC® 90, available from Penwest Pharmaceutical Co., Patterson, N.J. Silicon dioxide, in addition to that provided by the silicified microcrystalline cellulose, may be added to the composition as a processing aid. For example, silicon dioxide can be included as a glidant to improve the flow of powder during compression in the manufacturing of solid dosage units, such as tablet.

In another embodiment, the carrier is at least a functional carrier such as buckwheat or spelt. By the addition of functional carriers into the composition, additional benefits may be provided such as lower glycemic index compared to standard carriers such as those mentioned above. Further, functional carriers can be allergen free (e.g., buckwheat), and by adding them into the production process, the botanical extracts of the invention may benefit from the flavonoids of these functional carriers, such as rutin and quercetin. Further, the high fiber content of these functional carriers may also facilitate and regulate intestinal transit. Finally, the added mineral benefit of selenium found in spelt may aid in metabolism.

The anti-inflammatory composition can include other inert ingredients, such as lubricants and/or glidants. Lubricants aid in the handling of tablets during manufacturing, such as during ejection from dies. Glidants improve powder flow during tablet compression. Stearic acid is an example of an acceptable lubricant/glidant.

The anti-inflammatory composition can be made in solid dosage form, such as tablets and capsules. This form provides a product that can be easily transported by an individual to a place of eating, such as a restaurant, and taken prior to, during, or after consumption of a foodstuff. The composition can be formulated into dosage units containing suitable amounts of the plant extract and/or active ingredient that permit an individual to determine an appropriate number of units to take based upon appropriate parameters, such as body weight, foodstuff size, or carbohydrate (e.g., sugar) content.

In further embodiments, a composition according to the present disclosure comprises an Anacardium extract enriched for flavans containing catechin, epicatechin, or a combination thereof. In certain embodiments, major active ingredients in an extract of Anacardium comprise flavan containing catechin, epicatechin, or a combination thereof, wherein the extract is enriched for these active ingredients from the testa.

In one embodiment, the botanical extract is present in the composition in a therapeutically effective amount, such as an amount of about 500.0 mg/kg or greater, preferably from about 500.0 mg/kg to about 2000.0 mg/kg, more preferably from about 1000.0 mg/kg to about 2000.0 mg/kg. The composition can be administered, for example, in a dosage of from about 500.00 mg/kg to about 2000.0 mg/kg per day of the plant extract for human equivalent dosing. The composition can be administered as a single dose, or in multiple doses. In one example, the compound is administered in up to three doses per day. For example, the compound may be administered prior to a meal, during a meal, or after a meal. In one embodiment, the composition is a dietary supplement having anti-inflammatory properties containing cashew testa extract in a therapeutically effective amount.

The dosage can be chosen to provide a level of inhibitory effect in a single unit that may be effective for some individuals and/or some foodstuffs, while also allowing for relatively simple dosage increases to provide other levels of inhibitory effects that can be effective for other individuals and/or other foodstuffs.

The inhibiting composition can be in a form adapted for oral ingestion. This form can be configured as a single dosage form intended to provide a specified dose of the plant extract. For example, the single dosage form can be a powder, a pill, a tablet, a capsule, or a drink shot. The single dosage form can include, for example, from about 500.0 mg/kg to about 2000.0 mg/kg of the plant extract for human equivalent dosing.

### EXAMPLES

### Examples—Materials and Chemical Profiling

### Example 1—Total Catechin (Flavanols) and Polyphenol Quantification of Raw (Pre-Extraction) Cashew Testa Material

Quantification of flavanols were performed by HPLC with the results presented in the following Table 1—

| Total Catechin | Catechin equivalents |
|---|---|
| Catechin | 43.4 mg/g |
| Epicatechin | 40.1 mg/g |

In weight percentage, total catechin content of the cashew testa raw material was 7.000%, based on total weight of the raw material.

Total polyphenols (anthocyanins, flavanols, hydroxycinnamic acids, and soluble proanthocyanidins) can be quantified by the method of Folin-Ciocalteu. Gallic acid is generally recognized as the reference standard of choice, and thus total polyphenol results are reported as gallic acid equivalents.

A stock solution of gallic acid (1 mg/mL) was serially diluted and used to generate standard curves for the estimation of total polyphenols. The sample cashew testa and gallic acid standards were added to a 96 well plate alone with diluted folin reagent (7% in water) and allowed to incubate at room temperature for 10 minutes, followed by addition of 200 g/L Na₂CO₃. After shaking, the 96 well plate was incubated at 40° C. for 20 minutes, and then analyzed at 755 nm by spectrophotometry.

The quantification of total polyphenols was performed by UV-Vis spectroscopy at 755 nm wavelength. Quantification of total polyphenols by the method of Folin-Ciocalteu resulted in total polyphenols of 1420 mg/g, expressed as gallic acid equivalents (mg/g). In weight percentage, total polyphenol content of the cashew testa raw material was approximately 25.000%, based on total weight of the raw material.

### Example 2—Preparation of 70% Ethanol Extracts from Cashew Testa

Dried cashew testa powder (Anacardium occidentale L.) (60 g) was loaded into three 100 ml stainless steel tubes and extracted twice using a solvent of 70% ethanol in DI water with a Thermo Scientific™ Dionex™ ASE 350 Accelerated Solvent Extractor at a temperature of 80° C. and pressure of 1500 psi. The extract solution was filtered and collected. The combined ethanol extract solution was evaporated with a rotary evaporator under vacuum to give a crude cashew testa extract.

The extraction results are provided in the following Table 2—

TABLE 2
Extraction of cashew testa

| | Plant | Extract | Extraction |
|---|---|---|---|
| Plant Part | Powder (g) | Weight (g) | Yield (wt %) |
| Testa | 60 | 23.78 | 39.63% |

### Example 3—Catechin Quantification of Cashew Testa Extract

Free catechins present in the cashew testa extract were determined using a C18 reversed-phase column (Luna® 5 μm C18(2) 100 Å LC Column 250×4.6 mm, available from Phenomenex®, Torrance, California, US) together with a Hitachi high performance liquid chromatograph with photodiode array detector (‘HPLC/PDA’). For mobile phase A, the solvent was 0.10% phosphoric acid (‘H₃PO₄’) in water, and for mobile phase B, the solvent B was acetonitrile (‘ACN’), which was used for elution at a flow rated of 1.0 ml/min with UV absorbance at 275 nm and a column temperature of 35° C. Catechin reference standards used were from Sigma-Aldrich Co. The reference standards were dissolved in methanol (‘MeOH’): 0.1% H₃PO₄ (1:1 ratio) with catechin (C1251) at a concentration of 0.5 mg/ml and epicatechin (E1753) at 0.1 mg/ml. Testing samples were prepared at 2 mg/ml in 50% MeOH in 0.1% H₃PO₄ in a volumetric flask and sonicated until dissolved (approximately 10 minutes), and then cooled to room temperature, mixed well, and filtered through a 0.45 μm nylon syringe filter. HPLC analysis was performed by injecting a 20 μl sample into the HPLC. Table 2 below provides the gradient table of HPLC analytical method—

TABLE 2
Gradient Table of HPLC Analytical Method

| Time (min) | Mobile Phase A | Mobile Phase B |
|---|---|---|
| 0.0 | 85.0 | 15.0 |
| 7.0 | 85.0 | 15.0 |
| 12.0 | 10.0 | 90.0 |
| 16.5 | 10.0 | 90.0 |
| 16.6 | 85.0 | 15.0 |
| 24.0 | 85.0 | 15.0 |

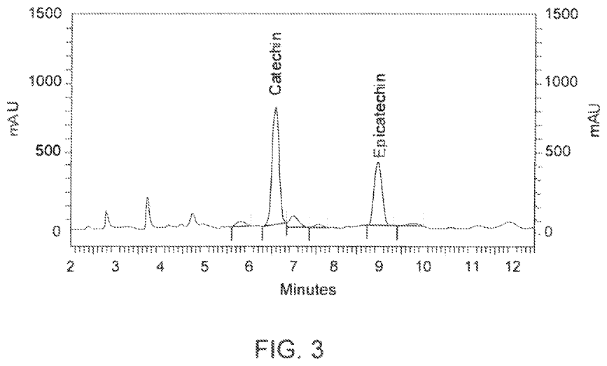

HPLC Catechin quantification results in cashew testa extract provided a catechin content of 9.40% and an epicatechin content of 6.12%, for a total catechin content of 15.52% by weight, based on total weight of the extract. Accordingly, the cashew testa extract can be standardized to a total catechin content of about 15.00% or greater by weight, based on total weight of the extract. The HPLC chromatogram for cashew testa extract at 275 nm wavelength is provided in FIG. 3. As shown in Example 1 above, the total catechin content of the raw cashew testa extract was only about 7.00% by weight, based on total weight of the raw material. Therefore, the cashew testa extract according to the present invention is enriched for one or more flavans, particularly total catechin. In another aspect, the cashew testa extract is enriched for catechin and epicatechin.

Total polyphenols in the cashew testa extract was about 55.00% by weight, based on total weight of the extract. Accordingly, the cashew testa extract according to the present invention is enriched for total polyphenols.

### Example 4—Chemistry Profiling of Cashew Testa Extract

Flavonoid compounds present in the cashew testa extract were determined using ultra high-pressure liquid chromatography (‘HPLC’) and mass spectrometry (ACQUITY® UPLC I-Class and XEVO® GS-XT-QTof system, both available from Water Corporation, Milford, Massachusetts USA). Column used was an ACQUITY® UPLC HSS T3 2.1×100 mm, 1.8 μm, with a column temperature of 40° C. and a sample temperature of 15° C. For the mobile phase, Solvent A was 10% acetonitrile (‘ACN’) in water (0.1% Formic Acid), and Solvent B was ACN. The acquisition range was 100-1500 Daltons (‘Da’), and the acquisition mode was electrospray ionization (‘ESI’) (-). Table 3 below provides the HPLC conditions—

TABLE 3
HPLC conditions for analyzing cashew testa extract

| Run Time (min) | Injection Volume (μL) | Concentration |
|---|---|---|
| 20.00 | 2.00 | 1 mg/mL |

Peak identification was based on accurate mass only. Flavan-3-ols digalloyl catechin, catechin and epicatechin were identified as the major components for cashew testa extract, having the following general structures—

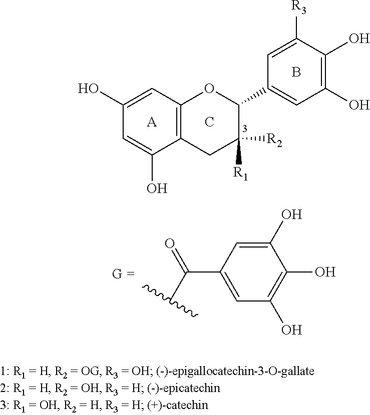

Procyanidin flavonoids were detected in the extract as well, including A- and B-type procyanidins, procyanidin tetramer, and procyanidin trimer, with B-type procyanidins being the major component of the procyanidins.

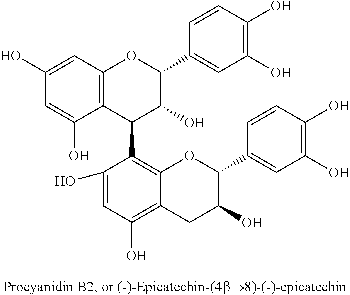 [SMILES: Oc1cc(O)c2c(c1)O[C@H](c1ccc(O)c(O)c1)[C@H](O)[C@H]2c1c(O)cc(O)c2c1O[C@H](c1ccc(O)c(O)c1)[C@@H](O)C2]

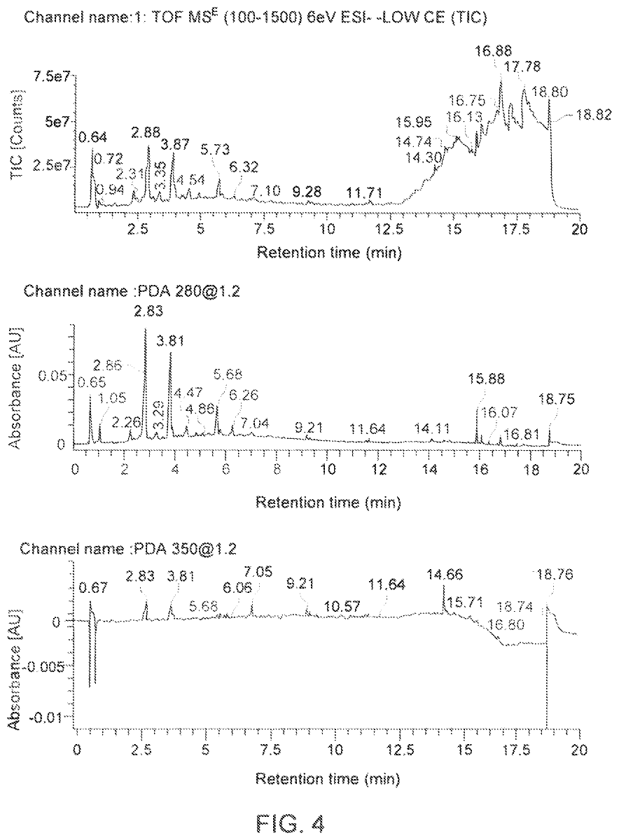

Compounds identified, in addition to those just mentioned, included vaccihein A, 6″-p-coumaroylprunin, and dunalianoside B, among others. LC/MS and LC/PDA chromatograms of cashew testa extract obtained from the analysis are illustrated in FIG. 4.

### Examples 5-7—In Vitro Bioassays

Extracts of cashew testa were prepared with food-grade ethanol, and then filtered and dried as described above. Research grade reagents were used for the rest of the assay preparations. Extracts were dissolved in dimethyl sulfoxide (‘DMSO’) to a final concentration of 50 mg/mL, and then diluted in appropriate buffer for each bioassay to working concentrations.

### Example 5—COX-1 and COX-2 Inhibition

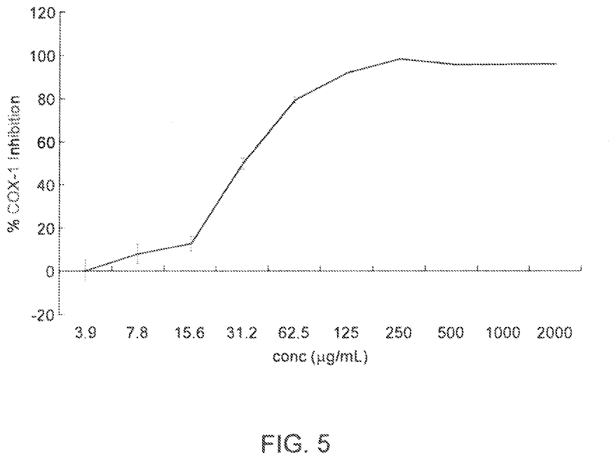

Cashew testa extract was tested for COX-1 inhibition using the cyclooxygenase-1 (‘COX-1’) Inhibitor Screening Kit (catalog #K548) from BioVision (Milpitas, California, US). This screening kit measures the production of the organic peroxide prostaglandin G2, a product generated by the COX enzyme, over a time course. Extracts were dissolved to working concentrations in DMSO with COX Assay Buffer to a final concentration of 5% DMSO. SC-560 COX-1 inhibitor was used as a positive control. COX-1 enzyme was reconstituted in sterile water and stored at −80° C. COX cofactor and arachidonic acid solutions were diluted just prior to use. COX probe, COX cofactor, and COX-1 enzyme solution were added to the test samples and controls before the arachidonic acid solution was quickly added to start the reaction. Fluorescence was measured every minute for 10 minutes at the following wavelengths: excitation −535 nm, emission 590 nm. The slope of the linear portion of the curve (FIG. 5) was deduced and percent inhibition of the uninhibited control was calculated. Referring to FIG. 5, various degrees of COX-1 inhibition were observed, depending on the concentration of cashew testa extract. Cashew testa extract COX-1 inhibition was observed to be from about 4 μg/mL to at least about 2000 μg/mL, more particularly from about 15 μg/mL to about 250 μg/mL, with an IC₅₀ of 32 μg/mL.

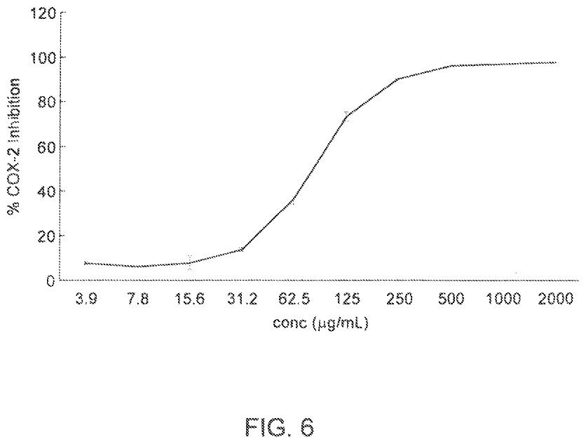

Cashew testa extract was tested for COX-2 inhibition using the cyclooxygenase-2 (‘COX-2’) Inhibitor Screening Kit (catalog #K547) from BioVision (Milpitas, California, US). This screening kit measures the production of the organic peroxide prostaglandin G2, a product generated by the COX enzyme, over a time course. Extracts were dissolved to working concentrations in DMSO with COX Assay Buffer to a final concentration of 10% DMSO. Celecoxib nonsteroidal anti-inflammatory drug (‘NSAID’) was used as a positive control. COX-2 enzyme was reconstituted in sterile water and stored at −80° C. COX cofactor and arachidonic acid solutions were diluted just prior to use. COX probe, COX cofactor, and COX-1 enzyme solution were added to the test samples and controls before the arachidonic acid solution was quickly added to start the reaction. Fluorescence was measured every minute for 10 minutes at the following wavelengths: excitation −535 nm, emission 590 nm. The slope of the linear portion of the curve (FIG. 6) was deduced and percent inhibition of the uninhibited control was calculated. Referring to FIG. 6, various degrees of COX-2 inhibition were observed, depending on the concentration of cashew testa extract. Cashew testa extract COX-2 inhibition was observed to be from about 4 μg/mL to at least about 2000 μg/mL, more particularly from about 30 μg/mL to about 250 μg/mL, with an IC₅₀ of 86 μg/mL. Accordingly, based on the results presented herein, cashew testa extract may have reasonable activities in ameliorating the activity or release of COX-1 and COX-2, suggesting its usage in inflammatory diseases mediated by COX-1 and COX-2.

### Example 6-5-LOX Inhibition

Cashew testa extract was tested for 5-LOX inhibition using the Lipoxygenase Inhibitor Screening Assay Kit (available from Cayman Chemical, Ann Arbor, Michigan, US) and potato 5-Lipoxygenase enzyme (available from Cayman Chemical). This kit measures hydroperoxides produced in the lipoxygenation reaction.

The extracts were dissolved in methanol to final working concentrations. 5-LOX enzyme, Chromagen, and Linoleic Acid solutions were prepared immediately before use. Nordihydroguaiaretic acid (‘NDGA’) was used as a positive control. 5-LOX enzyme was added to the test samples and controls and incubated for five minutes at room temperature to allow for enzyme/inhibitor interaction. Linoleic acid substrate was added to the plate to initiate the reaction, and the plate was then shaken at room temperature for 10 minutes. Chromagen was added to visualize the hydroperoxides formed during the reaction and the plate was shaken at room temperature for another five minutes. The absorbance was then read at 492 nm. Percent inhibition of the extract concentration was calculated in comparison to the uninhibited control wells.

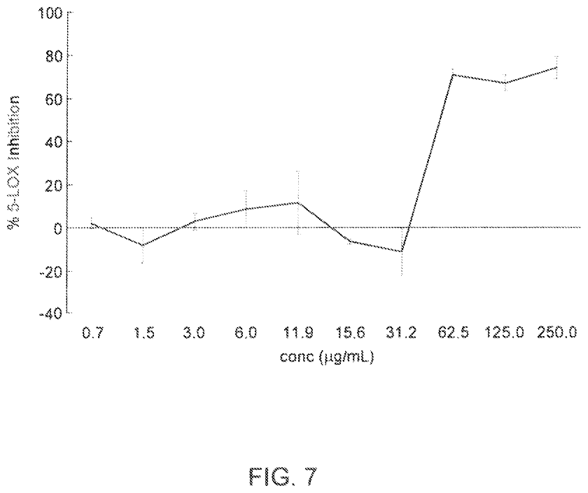

Cashew testa extract was tested for its 5-LOX inhibition activity at 10 different concentrations (0.7, 1.5, 3.0, 6.0, 11.9, 15.6, 31.2, 62.5, 125.0 and 250.0 μg/mL). NDGA was used as a positive control at 100 μM with a 100% 5-LOX enzyme inhibition. Referring to FIG. 7, cashew testa extract 5-LOX inhibition was observed to be from about 32 μg/mL to at least about 250 μg/mL, more particularly from about 32 μg/mL to about 125 μg/mL, with an IC₅₀ of 55 μg/mL observed for the cashew testa extract. Accordingly, based on the results presented herein, cashew testa extract may have reasonable activities in ameliorating the activity or release of 5-LOX, suggesting its usage in inflammatory diseases mediated by 5-LOX.

### Example 7—HMGB1 Inhibition

HMGB1 Experimental Procedure—

Cell Culture. Murine macrophage-like cells (available as RAW 264.7 (ATCC® TIB-71™) from American Type Culture Collection (ATCC), Manassas, Virginia, US) were cultured in Dulbecco's Modified Eagle's Medium (‘DMEM’) ((DMEM) (ATCC® 30-2002™), from American Type Culture Collection (ATCC), Manassas, Virginia, US) supplemented with 10% fetal bovine serum (from Atlanta Biologicals, Lawrenceville, Georgia, US). The cells were maintained under normoxic conditions (5% CO_{2/21}% 02), allowed to grow to 70-80% confluency, and subcultured every two (2) days.

Extract/Drug Preparation. Cashew testa extract was stored in powder form at −20° C. Prior to treating cells with extract, a stock solution volume of the extract was adjusted to a final concentration of 50 mg/mL in dimethyl sulfoxide (‘DMSO’) (from AMRESCO, Inc., Solon, Ohio, US) and stored at −20° C. Extract was diluted to a final concentration of 0.25 mg/mL in serum-free Opti-MEM™ I medium (from Gibco-BRL, Gaithersburg, Maryland, US) and filtered sterilized by 0.2 μm PES syringe filter (from VWR, Radnor, Pennsylvania, US). Sodium salicylate (from AMRESCO, Inc., Solon, Ohio, US) was prepared at 2-20 μM as a positive control, which can attenuate hyperoxia-induced HMGB1 release from macrophages.

Hyperoxia Exposure. The exposure of murine macrophage RAW 264.7 cells to hyperoxia was achieved in sealed, humidified Plexiglas chambers (from Billups-Rothenberg, Del Mar, California, US) flushed with 95% O₂/5% CO₂ at 37° C. for 24 hours.

HMGB1 ELISA. To determine the levels of extracellular HMGB1, RAW 264.7 cells were cultured in serum-free Opti-MEM™ I medium (from Gibco-BRL, Gaithersburg, Maryland, US) in 6-well plates and were kept at either 21% 02 (room air) or exposed to 95% 02 with or without the cashew testa extract for 24 hours. After hyperoxic exposure, the levels of HMGB1 in the culture media were measured by ELISA (enzyme-linked immunosorbent assay). Cell culture media was collected and pelleted at 500 g for 5 minutes at 4° C. Equal volumes of cell culture supernatant were then approximately 6-x's concentrated using Amicon Ultra-4 centrifugal units (from EMD Millipore, Burlington, Massachusetts, US). Just after concentration, equal volumes of cell culture supernatant concentrate were loaded onto a 96-well plate for determination of HMGB1 by ELISA according to manufacturer's instructions (from Chondrex, Inc., Redmond, Washington, US). Plate absorbances were determined by reading the optical density (‘OD’) value at 450 nm (with 630 nm used as a reference) on a Thermo Multiscan Ex microplate reader (from Thermo Scientific, Waltham, Massachusetts, US). HMGB1 levels were determined in sample cell culture supernatant by comparison to a standard curve and further corrected by applying concentration factors.

Statistical Analysis. Data was presented as the mean±standard error of the mean (SEM) of one to three independent experiments. Data was analyzed by use of one-way analysis of variance (ANOVA) using Fisher's Least Significant Difference (‘LSD’) post-hoc analysis and GraphPad Prism version 6 software (from GraftPad Software, La Jolla, California, US). A P-Value of <0.05 was considered statistically significant.

HMGB1 Experimental Results—

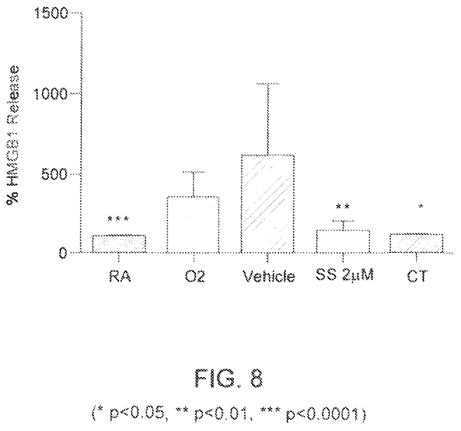

Referring to FIG. 8, it is seen that hyperoxia (‘02’) resulted in a significant increase in HMGB1 level compared to cells treated with 21% 02 (‘RA’). These elevated levels of HMGB1 were reduced closer to normal levels (cells exposed to room air (RA) with no treatment) because of treatment with cashew testa extract (‘CT’). Similar reductions were observed for the positive control sodium salicylate (‘SS’). Reductions for both treatment groups (SS and CT) were statistically significant. Accordingly, based on the results presented herein, cashew testa extract may have reasonable activities in ameliorating the activity or release of HMGB1, suggesting its usage in inflammatory diseases mediated by HMGB1.

The above data illustrates that the botanical extract of the testa of Anacardium occidentale L. has one or more compounds that exhibit anti-inflammatory activity. More particularly, the cashew testa extract may have reasonable activities in ameliorating the activity or release of COX-1, COX-2, 5-LOX, and/or HMGB1.

### Example 8—Efficacy of Anacardium occidentale L. Extract in Collagen-Induced Rat Paw Arthritis Induction

The collagen-induced arthritis (CIA) model in rats is the most commonly studied autoimmune model of rheumatoid arthritis (RA), with several pathological features resembling the immune mediated polyarthritis in human RA. Its shortest duration between immunization and disease manifestations makes the model more feasible for therapeutic efficacy evaluations. During its pathophysiology, following inoculation of heterogenic type II collagen (CII) from bovine nasal septum, rats will mount both humoral and cellular responses for the antigen. This sensitization subsequently will lead the host to fail to recognize self and to attack its own type II collagen where exclusively present in the joint cartilages. Upon induction, rats will experience inflammatory pain and swelling, cartilage degradation, synovial hyperplasia, panus formation, mononuclear cell infiltration, deformity, and immobility. Therefore, this model is ideal to evaluate the effectiveness of cashew testa extract administered orally at low, mid and high doses in reducing signs and symptoms associated with arthritis.

Cartilage is the main component of articular structure and consists of chondrocytes that are embedded in a dense and highly organized extracellular matrix (‘ECM’). ECM is synthesized by the chondrocytes and is composed of a collagenous network that primarily contains type II collagen, along with glycosaminoglycans (‘GAGs’) and associated proteoglycans. While the exact pathological sequences are unknown, all structural components of the joint are involved in the pathogenesis of arthritis. Along with aggrecan breakdown, degradation of collagen is a central feature of arthritis. Pro-inflammatory cytokines such as tumor necrosis factor (‘TNF’)-α and interleukin (‘IL’)-1β are known to play important roles in cartilage matrix degradation in the articular cartilage through a cascade of events that lead to stimulation of aggrecanase and matrix metalloproteinase (such as MMP13) production. While TNF-α is known as the driving force for the inflammatory process, IL-1β is believed to orchestrate recruiting other proinflammatory cytokines and chemokines. Together they can amplify, sustain, and perpetuate the disease process. Degraded cartilage is one of the major clinical manifestations in both rheumatoid arthritis (‘RA’) and osteoarthritis (‘OA’). In particular, Urinary C-terminal telopeptide of type II collagen (‘μCTX-II’) has been by far the most studied and frequently referred to biomarker of cartilage degradation that can be used for the purpose of diagnosis, determination severity of disease or prediction disease progression, prognosis and monitoring efficacy of treatment. As a result, suppression of any of these mediators could have therapeutic advantage in OA/RA. It is also noted that at the early stages of arthritis, there is an effort by the chondrocyte to rebuild and replenish the degrading extra cellular matrix such as collagen and aggrecan. This anabolic property can be assessed by measuring the serum level of PIIANP representing collagen synthesis.

Collagen induced arthritis (CIA) in rats was developed and utilized to evaluate efficacy of orally administered cashew skin extract for three weeks post disease induction. The study included seven groups of rats (n=9 rats/group). The rats were purpose bred male Sprague—Dawley rats (7-8 weeks old, Charles River Laboratories Inc., Wilmington, MA). Animals were acclimated for two weeks to achieve the required body weight before being assigned randomly to their respective group. Rats (3/cage) were housed in a polypropylene cage and individually identified by numbers on their tail. Individual cages were identified with a cage card indicating project number, test article, dose level, group, and animal number. Harlan Soft cob bedding (Envigo Tekland 7087, Envigo, Indianapolis, Indiana) was used and changed at least twice/week. Animals were provided with fresh water and rodent chow diet (Teklad 2018, Envigo, Indianapolis, Indiana) ad libitum and housed in a temperature-controlled room (22.2° C.) on a 12-h light-dark cycle throughout the study.

The study rats were randomized and administered one of seven study articles as noted in Table 4—

TABLE 4

| | Group | N (no. of rats) | Dose (mg/kg) |
|---|---|---|---|
| G1 | Control + vehicle (0.5% CMC *) | 9 | 0.0 |
| G2 | CIA + vehicle (0.5% CMC) | 9 | 0.0 |
| G3 | CIA + MTX | 9 | 0.5 |
| G4 | CIA + low dose CNT | 9 | 50.0 |
| G5 | CIA + mid dose CNT | 9 | 100.0 |
| G6 | CIA + high dose CNT | 9 | 200.0 |
| G7 | Glucosamine + Chondroitin | 9 | 150.0 G + |
| | | | 120.0 C |

* CMC = carboxymethylcellulose; MTX = Methotrexate; CNT = cashew testa extract; G = glucosamine; C = chondroitin. Dosing based on Nair, A. B. et al., J BASIC CLIN PHARM, “A simple practice guide for dose conversion between animals and human”, March 2016, Vol. 7, No. 2, pp. 27-31.

Anacardium occidentale L. extract used in the CIA study was prepared as described in Example 1 above and quantified for total catechin content according to Example 2, with the extract having a total catechin content of 18.4% by total weight of the extract.

As noted in Table 4 above, the rats were randomized into seven treatment groups, with nine (9) rats in each group, based on their body weight. On treatment start day, the average body weight of the rats was 189.7±11.7 g. The animals were orally treated according to Table 4 with Methotrexate, cashew skin extract at three dosages, and glucosamine and chondroitin daily for three weeks. Methotrexate is an effective immunosuppressant optimally used to treat autoimmune mediated arthritis such as CIA rats. The normal control rats and CIA rats were treated with the carrier vehicle (0.5% Carboxymethyl cellulose) only.

For two (2) weeks prior to induction, the rats were gavaged with freshly prepared respective test materials suspended in 0.5% CMC at 10 ml/kg/rat. Samples in solution were vortexed before oral administration to maintain the homogeneity of test materials. Ankle diameter, paw thickness and pain sensitivity measurements were taken before induction of arthritis at priming for baseline.

For induction, collagen type-II from bovine nasal septum (Elastin Products Company, Owensville, Michigan) and Incomplete Freund's adjuvant (‘IFA’, from Sigma, St. Louis, Missouri) were used. All materials were kept at suitable temperature as recommended by the manufacturer. At the time of preparation, 60 mg of collagen was weighed and added to a pre-chilled 15 ml 0.1M acetic acid in a 60 ml size flask with a magnetic stirrer to yield 4 mg/ml concentration. The mixture was dissolved by gently stirring overnight at 4° C. The next morning, the dissolved collagen was emulsified with equal volume of IFA (15 ml) to achieve a final concentration of 2 mg/ml Collagen. Rats sedated with isoflurane were then primed intradermal with 400 μl of the emulsified collagen at the base of their tail at two sites using a 1 ml syringe fitted in 26 g needle. The dissolved mixture was kept in ice bucket and stirred between groups at the time of injection to preserve uniform consistency.

Post-induction, all the rats continued receiving their respective treatment materials after priming. The rats received a total of 5 weeks of treatment (i.e., 2 weeks before induction and 3 weeks after induction).

Rats were evaluated for arthritis index before injection of a booster dose. Inoculated a booster dose of 2 mg/ml type II collagen emulsified with equal volume of IFA at 100 μI/rat/site, following the same preparation as indicated in the pre-induction treatment. Paw thickness, ankle diameter and pain sensitivity measurements were taken before injection of the antigen on day 7.

During the in-life period, arthritis severity index, paw thickness, ankle diameter and pain sensitivity were monitored. Urine and serum were collected at the end of study for biomarker analysis. Necropsy was carried out on day 22 for all the groups post priming. At necropsy, the ankle joint from each rat was collected for histopathology analysis. Urinary cartilage degradation marker (CTX-II), proinflammatory cytokines (TNF-α, IL-1β, and IL-6), cartilage synthesis marker (PIIANP) and matrix degrading proteases (MMP13) were measured.

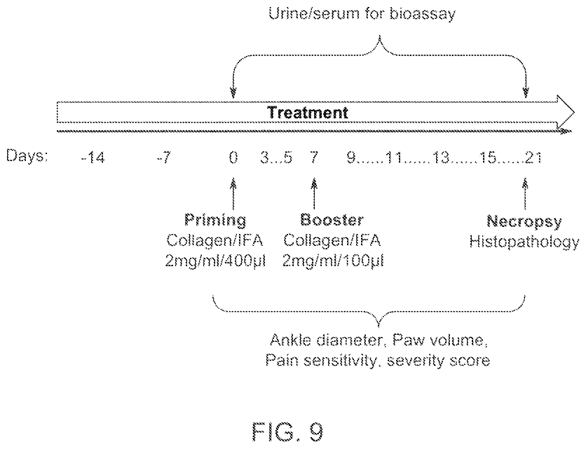

All animal experiments were conducted according to institutional guidelines congruent with the guide for the care and use of laboratory animals, which were reviewed and approved by the Institutional Animal Care and Use Committees (IACUC) (Approval number IA-P02-092619). Experimental design is depicted in FIG. 9.

Clinical findings such as arthritis severity index, paw thickness, ankle diameter and pain sensitivity data have been compiled as follows.

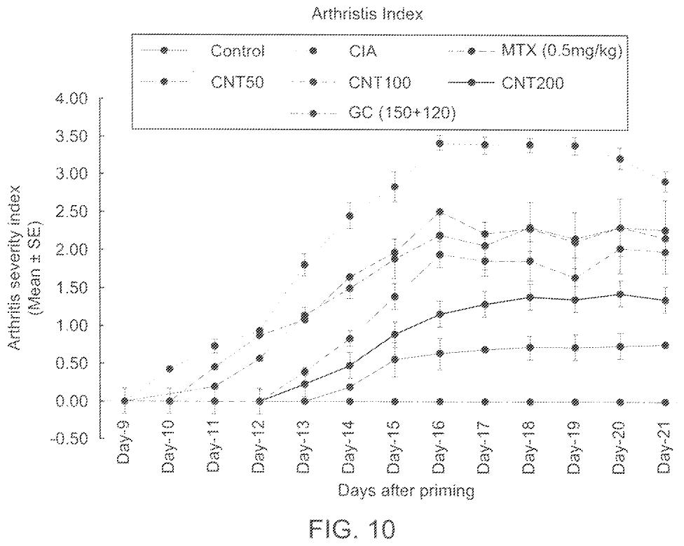

Arthritis severity index. Rats continued to show a slow progression of disease for the duration of study. As seen in the data below, rats treated with all the treatment groups showed various degrees of severity inhibition. In particular, rats treated with 100 mg/kg and 200 mg/kg CNT showed statistically significant suppression in arthritis severity from day 10 and continued this significance for the duration of study (FIG. 10, Table 5). While the vehicle treated rats showed arthritis symptoms on day 10 after priming, rats treated with the 100 mg/kg and 200 mg/kg CNT started the symptom on day 13 after priming (i.e., a 72-hour delay in the onset of arthritis symptoms). The 50 mg/kg CNT and the 150 mg/kg G+120 mg/kg C group started the arthritis symptom on day 11 after priming. For comparison, disease onset for the Methotrexate group was day 14 after priming. For all Figures illustrating the area under the curve, the linear trapezoid rule was used to calculate the area under the curve for days 9-21. % inhibition={(mean value of treatment-mean value of CIA+)/(mean value of control-mean value of CIA+)}*100.

TABLE 5
P-values for arthritis severity index compared to vehicle-treated CIA+

| | P-value | | | | | | | | | | | |
|---|---|---|---|---|---|---|---|---|---|---|---|---|
| Group | Day 10 | Day 11 | Day 12 | Day 13 | Day 14 | Day 15 | Day 16 | Day 17 | Day 18 | Day 19 | Day 20 | Day 21 |
| Control | 0.0042 | 0.0002 | 0.0005 | 0.0000 | 0.0000 | 0.0000 | 0.0000 | 0.0000 | 0.0000 | 0.0000 | 0.0000 | 0.0000 |
| MTX | 0.0042 | 0.0002 | 0.0005 | 0.0000 | 0.0000 | 0.0000 | 0.0000 | 0.0000 | 0.0000 | 0.0000 | 0.0000 | 0.0000 |
| CNT 50 | 0.0178 | 0.0107 | 0.2163 | 0.0700 | 0.0141 | 0.0085 | 0.0035 | 0.0030 | 0.0074 | 0.0025 | 0.0370 | 0.1245 |
| CNT 100 | 0.0042 | 0.0002 | 0.0005 | 0.0000 | 0.0000 | 0.0002 | 0.0038 | 0.0041 | 0.0015 | 0.0001 | 0.0204 | 0.0726 |
| CNT 200 | 0.0042 | 0.0002 | 0.0005 | 0.0000 | 0.0000 | 0.0000 | 0.0000 | 0.0001 | 0.0000 | 0.0000 | 0.0002 | 0.0004 |
| GC(150 + 120) | 0.0042 | 0.2779 | 0.8382 | 0.0733 | 0.0472 | 0.0340 | 0.0775 | 0.0242 | 0.0225 | 0.0087 | 0.0742 | 0.1423 |

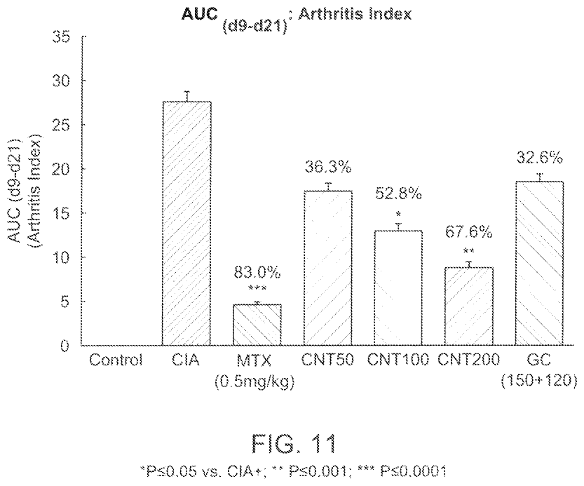

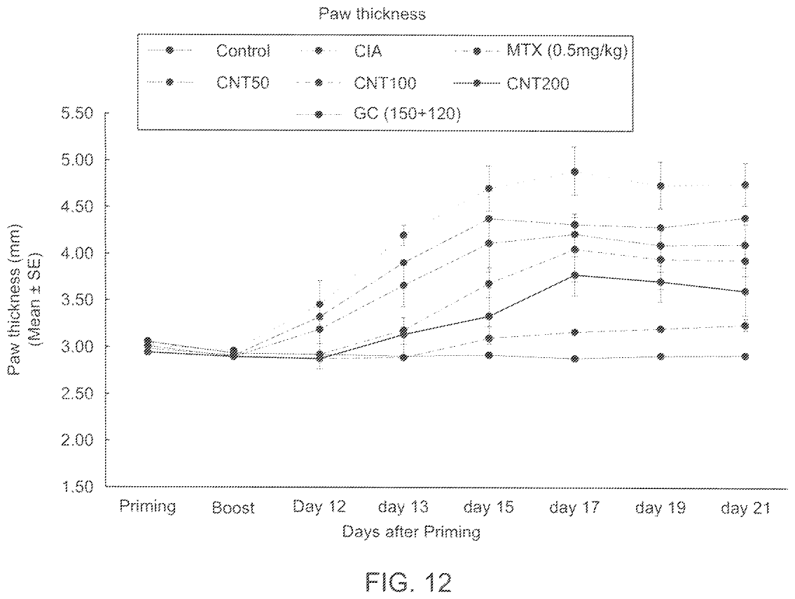

Paw thickness. In agreement with the severity score, rats treated with the 100 mg/kg and 200 mg/kg CNT showed statistically significant reduction in paw swelling starting from day 12 and maintained this significance for the duration of study (FIG. 11, Table 6). The 50 mg/kg CNT group showed a non-statistically significant but better reduction in paw thickness compared to the GC group. When the total area under the swelling curve (day 7-day 21) for these reductions were considered, rats showed statistically significant 55.8% and 68.7% reduction in paw edema compared to the vehicle treated CIA group for the 100 mg/k and 200 mg/kg CNT groups, respectively (FIG. 12). Percent reductions of 36.6% and 23.5% with P-values of 0.15 and 0.36, in paw edema were observed for rats treated with 50 mg/kg CNT and GC, respectively, compared to the vehicle treated CIA. The Methotrexate group showed 90.2% reduction.

TABLE 6
P-values for paw thickness compared to vehicle-treated CIA+

| | P-values | | | | | |
|---|---|---|---|---|---|---|
| Group | Day 12 | Day 13 | Day 15 | Day 17 | Day 19 | Day 21 |
| Control | 0.0572 | 0.0000 | 0.0000 | 0.0000 | 0.0000 | 0.0000 |
| MTX | 0.0388 | 0.0000 | 0.0000 | 0.0000 | 0.0000 | 0.0000 |
| CNT 50 | 0.3734 | 0.0575 | 0.1725 | 0.0862 | 0.0954 | 0.0702 |
| CNT 100 | 0.0438 | 0.0000 | 0.0061 | 0.0395 | 0.0380 | 0.0338 |
| CNT 200 | 0.0468 | 0.0000 | 0.0004 | 0.0278 | 0.0281 | 0.0014 |
| GC(150 + 120) | 0.6574 | 0.2100 | 0.4278 | 0.1508 | 0.2207 | 0.3370 |

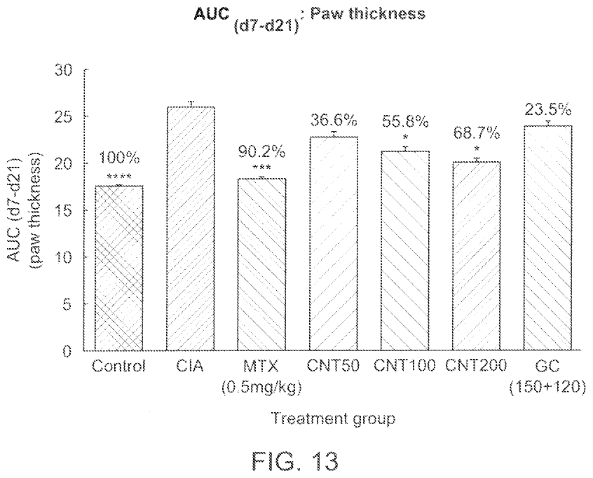

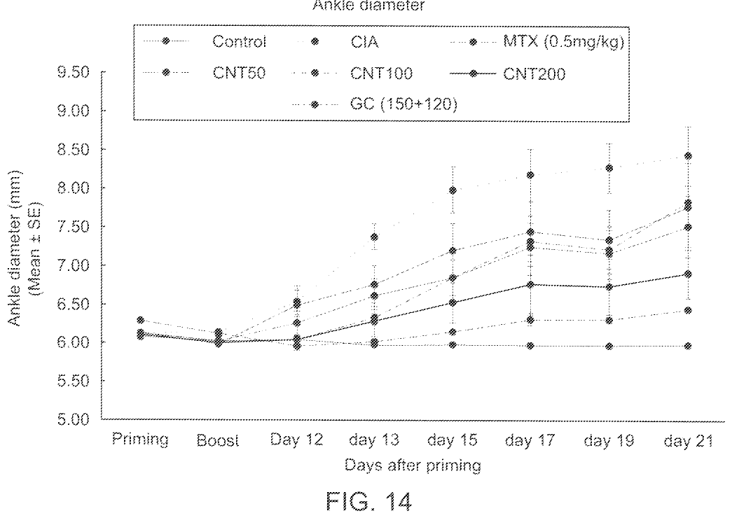

Ankle diameter. Statistically significant, similar pattern in reductions of ankle diameters were observed for rats treated with 100 mg/kg and 200 mg/kg CNT until day 15 post induction (FIG. 13, Table 7). Thereafter only the 200 mg/kg group showed statistically significant reductions in ankle diameter for the rest of the duration of study. Reductions in ankle diameter on days 17 and 21 were not statistically significant for the 100 mg/kg CNT group. Rats treated with 50 mg/kg CNT showed statistically significant reductions in ankle diameter on days 13, 15 and 19 post induction. In contrast, the GC treated rats experienced no significant reductions in ankle diameter in the course of study. Here, from the cashew testa extracts, only the 200 mg/kg CNT group showed statistically significant reductions (i.e., 70.3%) in ankle width when the area under the curve was considered for days 7 to 21. Statistically non-significant 49.3%, 50.9% and 36.2% reductions in ankle diameter were observed for the 50 mg/kg CNT, 100 mg/kg CNT and GC groups, respectively for the AUC. The Methotrexate group showed 89.1% reduction (FIG. 14).

TABLE 7
P-values for ankle diameter compared to vehicle treated CIA+

| | P-values | | | | | |
|---|---|---|---|---|---|---|
| Group | Day 12 | Day 13 | Day 15 | Day 17 | Day 19 | Day 21 |
| Control | 0.0196 | 0.0000 | 0.0000 | 0.0000 | 0.0000 | 0.0000 |
| MTX | 0.0312 | 0.0000 | 0.0000 | 0.0001 | 0.0000 | 0.0003 |
| CNT 50 | 0.3162 | 0.0228 | 0.0124 | 0.0754 | 0.0300 | 0.1462 |
| CNT 100 | 0.0373 | 0.0002 | 0.0115 | 0.1041 | 0.0375 | 0.3843 |
| CNT 200 | 0.0380 | 0.0011 | 0.0034 | 0.0184 | 0.0083 | 0.0116 |
| GC(150 + 120) | 0.8567 | 0.0815 | 0.4278 | 0.1508 | 0.2207 | 0.3370 |

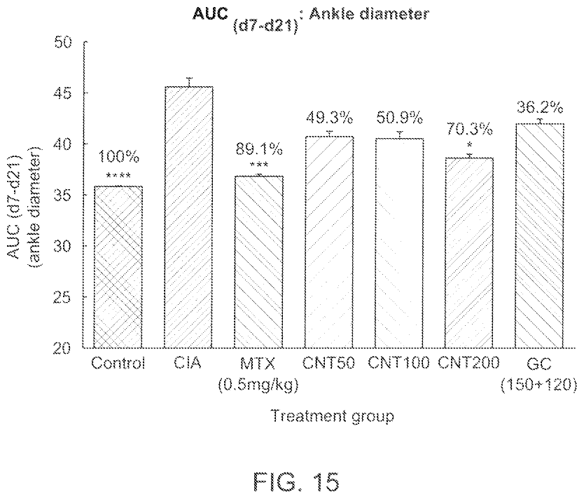

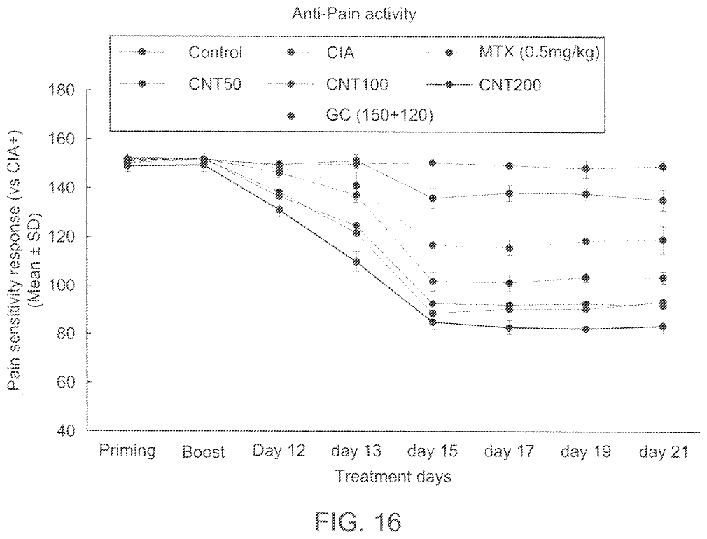

Pain sensitivity. Response to pressure as a measure of pain sensitivity was measured using a Randall—Selitto probe attached to an electronic monitor on priming day, boost, day 12, 13, 15, 17, 19 and 21. Both the left and right hind legs were monitored on those days, and their average was used for data analysis. Changes from the vehicle treated CIA rats have been reported as pain tolerance on those days. The highest pain tolerance was observed for rats in the Methotrexate (14.1-67.1% vs Vehicle treated CIA) group followed by the 200 mg/kg (13.5-43.8% vs Vehicle treated CIA) and the 100 mg/kg (11.8-25.8% vs Vehicle treated CIA) (FIGS. 15 and 16). Rats in the 50 mg/kg CNT and GC group showed similar reductions in pain sensitivity for all the time points monitored. Statistically significant pain inhibition was observed at all time points as of day-12 for all the groups when compared to vehicle treated CIA rats (Table 8).

TABLE 8
P-values for pain tolerance compared to vehicle treated CIA+

| | P-values | | | | | |
|---|---|---|---|---|---|---|
| Group | Day 12 | Day 13 | Day 15 | Day 17 | Day 19 | Day 21 |
| Control | 0.0000 | 0.0000 | 0.0000 | 0.0000 | 0.0000 | 0.0000 |
| MTX | 0.0000 | 0.0000 | 0.0000 | 0.0001 | 0.0000 | 0.0003 |
| CNT 50 | 0.0003 | 0.0228 | 0.0124 | 0.0754 | 0.0300 | 0.1462 |
| CNT 100 | 0.0000 | 0.0002 | 0.0115 | 0.1041 | 0.0375 | 0.3843 |
| CNT 200 | 0.0000 | 0.0011 | 0.0034 | 0.0184 | 0.0083 | 0.0116 |
| GC(150 + 120) | 0.0025 | 0.0815 | 0.4278 | 0.1508 | 0.2207 | 0.3370 |

### Biomarkers—

Urine CTX-II—

Assay. Rat urine samples were diluted 1:3 and the presence of CTX-II was measured using the Rat CTX-II ELISA kit from Mybiosource as follows. Diluted urine was added to a microplate coated with CTX-II antibody and allowed to bind for 2 hours at 37° C. A biotin-conjugated antibody against CTX-II was then added and allowed to bind to the CTX-II from the rat urine for 1 hour at 37° C. The microplate was washed thoroughly to remove unbound urine and antibody before an enzyme-conjugated avidin antibody was added to bind to the biotin-conjugated antibody for specific detection. The avidin antibody was allowed to bind for 1 hour at 37° C. Washing was repeated, enzyme substrate was added, and the plate was developed for 30 minutes at 37° C. After the addition of stop solution, the absorbance was read at 450 nm, multiplied by dilution factor, and the concentration of CTX-II calculated based on the absorbance readings of a CTX-II standard curve.

Normalization.

Creatine—CTX-II amount was normalized to the amount of Creatinine in the urine using a Creatinine Parameter Assay Kit (R&D Systems) as follows. Urine was diluted 1:20, mixed with alkaline picrate (5 parts 0.13% picric acid: 1 part 1 N NaOH) in a microplate, and incubated at room temperature for 30 minutes. Absorbance was read at 492 nm, and Creatinine amount in urine was calculated based on the absorbance readings of a Creatinine standard curve.

Protein—CTX-II amount was normalized to the amount of total protein in the urine using a Pierce BCA Protein Assay kit (ThermoFisher Scientific) as follows. The urine was diluted 1:20, mixed with bicinchoninic acid (BCA) reagent in a microplate, and incubated at 37° C. for 30 minutes. Absorbance was read at 580 nm, and protein concentration in the urine was calculated based on the absorbance readings of a bovine serum albumin standard curve.

Results.

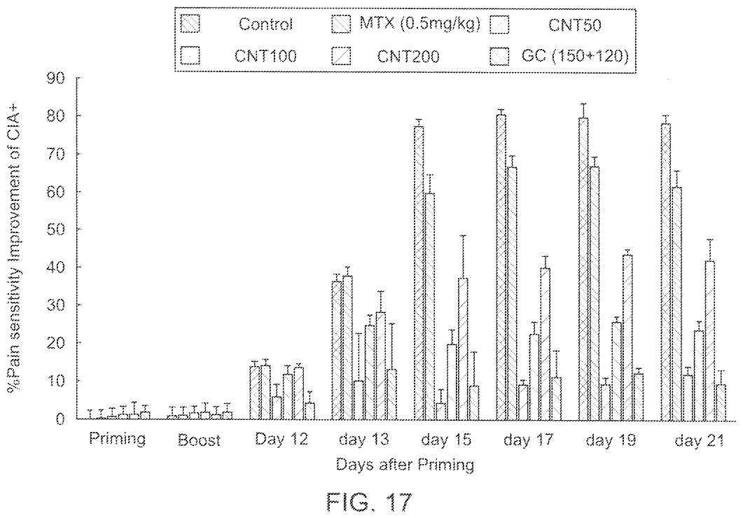

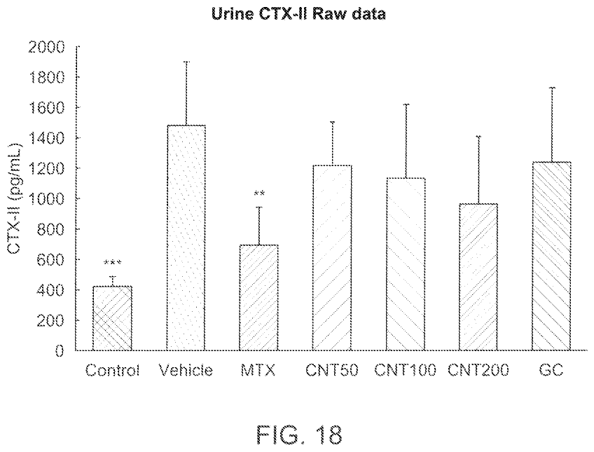

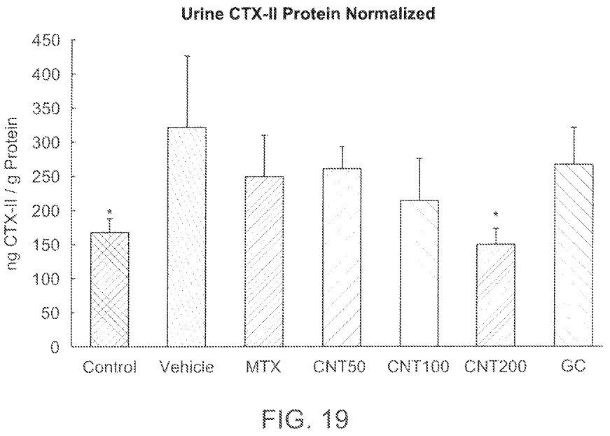

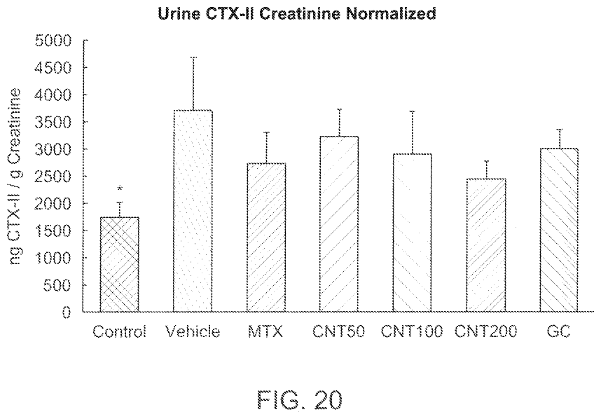

As illustrated in FIGS. 17-19, statistically significant increase (3.5-fold in the raw data, and 2-fold in the protein and creatinine normalized) in urinary CTX-II level was observed for vehicle-treated CIA rats compared to the normal control confirming severity of disease. In agreement with the clinical observation (arthritis severity, paw swelling and ankle diameter), rats treated with cashew testa extract showed dose correlated prevention of cartilage degradation. The highest inhibition in matrix breakdown was observed for rats treated with the high dose (200 mg/kg) of cashew testa extract, followed by the mid-dose (100 mg/kg). In fact, when values were normalized by protein (53.4% inhibition vs vehicle; p=0.04) or creatinine (33.0% inhibition; p=0.11), the percent inhibition values observed for the rats in the 200 mg/kg group were higher than any of the treatment groups. Methotrexate seemed to spare significant degradation of cartilage (up to 53.8% protection; P=0.005) compared to vehicle treated diseased CIA rats in the raw data. These values were moderate when they were normalized with protein (22.9%, p=0.37) and creatinine (27.2%; p=0.22) for the methotrexate group relative to vehicle treated CIA rats. Cashew testa extract administered at 100 mg/kg showed 23.2% (p=0.29), 33.0% (p=0.20) and 21.3% (p=0.36) cartilage protection for the raw data, protein normalized and creatinine normalized, respectively compared to the untreated CIA rats. Rats in the 50 mg/kg cashew testa extract and the GC group showed minimal cartilage protection. Reductions of 17.8%, 19.0% and 12.3% for the 50 mg/kg cashew testa extract, and 16.3%, 16.5% and 17.9% for the GC treatment group were observed in the raw data, protein normalized and creatinine normalized CTX-II, respectively.

Cytokines IL-1β/IL-6/TNF-α—

Sample collection. At completion of study, blood from cardiac puncture was collected for each animal. Blood was spun at 3000 rpm for 15 min. About 700-80011.1 of serum was isolated from each rat. Both samples were kept at −80° C. until use.

ELISA Assay. The presence of cytokines IL-1β/IL-6/TNF-α was measured using the Rat IL-1β/IL-6/TNF-α Quantikine ELISA kit (R&D Systems, Minneapolis, Minnesota) as follows. Undiluted serum was added to a microplate coated with polyclonal IL-1β/IL-6/TNF-α antibody and allowed to bind for 2 hours at room temperature. The microplate was washed thoroughly to remove unbound serum, and then a polyclonal enzyme-conjugated IL-1β/IL-6/TNF-α antibody was added and allowed to bind for 2 hours at room temperature. Washing was repeated, enzyme substrate was added, and the plate was developed for 30 minutes at room temperature. After the addition of stop solution, the absorbance was read at 450 nm and the concentration of IL-1β/IL-6/TNF-α calculated based on the absorbance readings of an IL-1β/IL-6/TNF-α standard curve.

Result for Serum IL-1β, IL-6, and TNF-α.

Proinflammatory cytokines such as IL-1β, TNF-α, and IL-6 play critical role alone or in concert in initiation, recruiting, progression and perpetuation of inflammation in the pathogenesis of OA/RA. Agents that reduce the level of these cytokines could mitigate the symptoms associated with OA/RA.

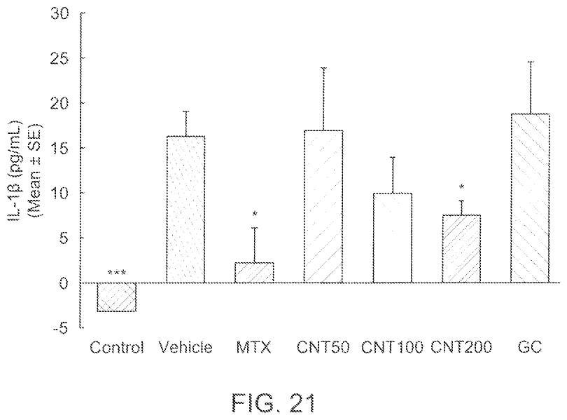

Referring to FIG. 21, statistically significant increase in serum IL-1β was observed for the CIA rats treated with vehicle (0.5% CMC). This increase in serum level of IL-1β was significantly reduced by the positive control Methotrexate (86.4% reduction, p=0.01) and 200 mg/kg CNT (53.1% reduction, p=0.01) compared to the CIA rats treated with vehicle. Rats in the 100 mg/kg CNT treated group showed a statistically non-significant 38.3% decrease in serum level of IL-1β. There was a non-significant 3.7% increase in serum IL-1β level for rats in the 50 mg/kg CNT group (FIG. 21).

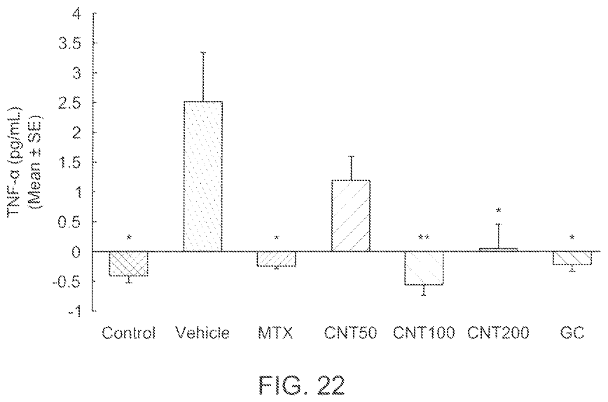

Similarly, the serum level of TNF-α was reduced as a result of cashew skin extract (FIG. 22). The level of TNF-α was found below 0 for the normal control and rats treated with Methotrexate, 100 mg/kg CNT and GC. These levels were statistically significant when compared to the vehicle treated CIA rats. Though the percent reductions for the 50 mg/kg and 200 mg/kg CNT were found as 52.1% (p=0.14 vs CIA rats treated with vehicle) and 98.3% (p=0.07 vs CIA rats treated with vehicle), respectively, when compared to the vehicle treated CIA rats, they failed to achieve statistical significance due to variations among individual rats.

Cashew testa extract treatment had no effect on rat serum IL-6 levels, with all serum values found below the blank. However, the IL-1β and TNFα data reflects what was observed in the in-life study clinical measurements, such as arthritis index, ankle diameter and paw thickness.

Type IIA Collagen N-Propetide (PIIANP)

Levels of PIIANP decrease in patients with OA and RA, suggesting that type IIA collagen synthesis may be altered in these diseases. Accordingly, the measurement of type IIA collagen synthesis based on this biomarker is useful in determining the efficacy of cashew testa extract in patients with joint diseases.

ELISA Assay. The presence of PIIANP was measured using the Rat Procollagen Type IIA N-Prop (PIIANP) ELISA kit (MyBiosource, San Diego, California) as follows. Undiluted serum was added to a microplate coated with PIIANP antibody as well as an HRP-conjugated PIIANP antibody and allowed to bind for one hour at 37° C. The microplate was thoroughly washed and a Chromagen solution was added and allowed to bind for 15 minutes at 37° C. After the addition of stop solution, the absorbance was read at 450 nm and the concentration of PIIANP calculated based on the absorbance readings of a PIIANP standard curve.

Results for Serum PIIANP

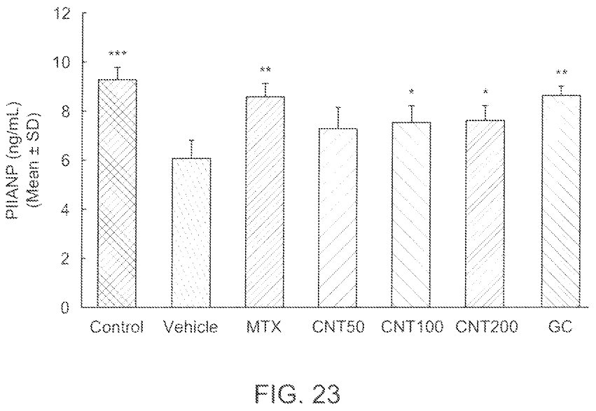

While the normal control rats showed a 53.0% increase in the level of serum PIIANP, a statistically significant decrease (34.7% vs. control) in serum PIIANP was observed for the CIA rats treated with vehicle compared to the control group (p=0.0002), indicating the induction of the model (see FIG. 23). In contrast, CIA rats treated with the positive control methotrexate had a significant increase in serum PIIANP (41%, p=0.001 compared to CIA+vehicle) when compared to the vehicle treated disease model. Rats in the cashew skin extract group showed a 20.6% (at 50 mg/kg), 25.3% (at 100 mg/kg) and 27.0% (at 200 mg/kg) increase in serum PIIANP compared to the vehicle treated CIA group. The increases observed for the mid-dose (100 mg/kg) and the high-dose (200 mg/kg) were statistically significant when compared to the vehicle treated CIA rats. These results indicate that the cashew skin extract treated rats have an increased amount of collagen being synthesized in response to the treatment. This shows that the treatments contribute to reversal of the collagen degradation phenotype that is characteristic of this animal model. At least in this category, the GC group showed statistically significant increase (44.3%, p=0.001) in PIIANP group when compared to the CIA rats treated with vehicle indicating cartilage regeneration activity.

Matrix Metalloproteinase 13 (MMP-13)

Matrix metalloproteinase 13 is a regulator of inflammation and is an enzyme that plays an important role in type II collagen degradation in articular cartilage in osteoarthritis. It also degrades proteoglycan, type IV and type IX collagen, osteonectin, and perlecan in cartilage.

ELISA Assay. The presence of MMP-13 in undiluted rat serum was measured using the MMP-13 Rat Matrix Metalloproteinase 13 (MMP-13) ELISA Kit (MyBioSource, San Diego, California) as follows. Undiluted serum was added to a microplate coated with MMP-13 antibody. After 2 hours at 37° C., MMP-13 in serum was bound to the plate and unbound serum was aspirated. A biotin-conjugated antibody specific for MMP-13 was added to the wells and allowed to bind for 1 hour at 37° C. The plate was thoroughly washed, and avidin conjugated Horseradish Peroxidase (HRP) was added to the plate. After 1 hour at 37° C., washing was repeated, and enzyme substrate was added to the plate. After developing for 20 minutes at 37° C., a stop solution was added, and the absorbance was read at 450 nm. The concentration of MMP-13 was calculated based on the absorbance readings of an MMP-13 standard curve.

Results for Serum MMP-13

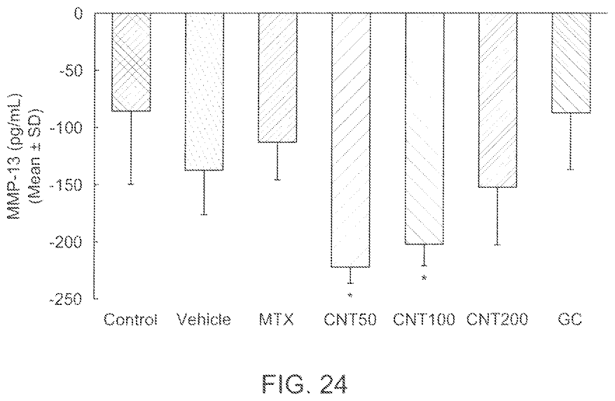

Referring to FIG. 24, it is seen that the serum MMP-13 concentrations were found lower than the standard curve for all the groups. Therefore, the results were inconclusive.

Histopathology

Procedures and Evaluations. At necropsy, the ankle joint was carefully dissected out, fixed in 10% buffered formalin, and sent to Nationwide Histology (Veradale, WA, USA) for further histopathology analysis. The fixed specimens were then decalcified with Calci-Clear Rapid for one and a half days and embedded in paraffin. Standardized 5 μm serial sections were obtained from each rat and stained with hematoxylin and eosin (HE) and Safranin 0-fast green to enable evaluation of proteoglycan content. A modified Mankin system (Mankin et al., 1981) was used to score structural and cellular alterations of articular components as indications of disease progression and/or treatment efficacy. The histological analysis was conducted by a certified Pathologist at Nationwide Histology.

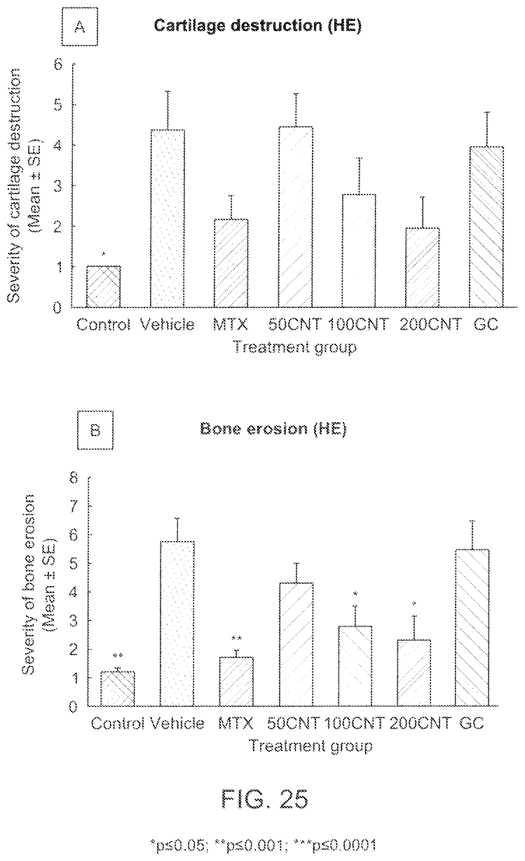

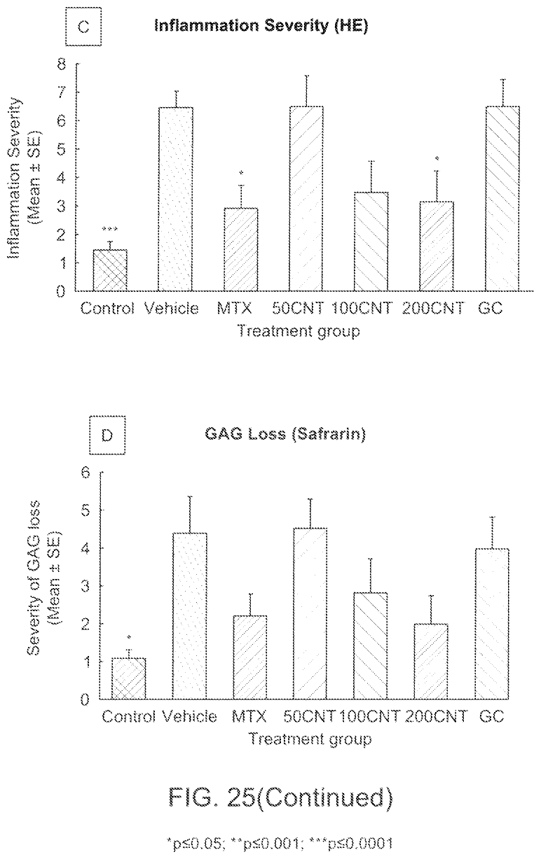

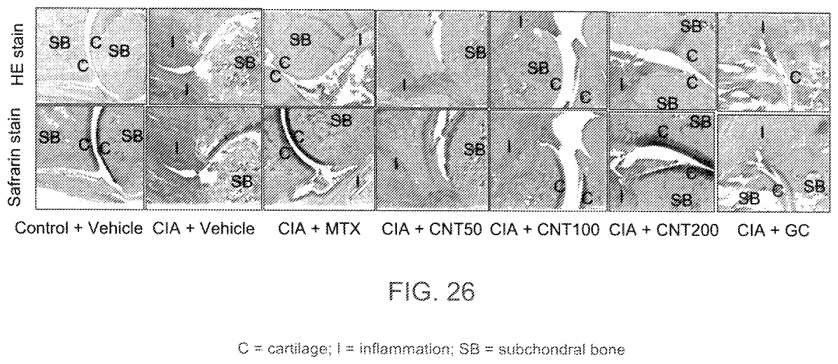

Results. The histopathology data were in alignment with the severity score of arthritis. When compared to the normal control rats, vehicle treated CIA rats showed severe synovitis, marked cartilage degradation, synovial hyperplasia, pannus formation and bone erosion (FIGS. 25 and 26). Vehicle treated CIA rats showed a 4.4-, 5-, 4.8- and 4.4-fold increase in severity of cartilage destruction, bone erosion, inflammation, and GAG loss, respectively, compared to normal controls. In contrast, rats treated with Methotrexate had relatively lower alternation in cartilage destruction (50.7% lower than vehicle), bone erosion (71.2% lower than vehicle), inflammation (55.8% lower than vehicle) and GAG loss (50.7% lower than vehicle) when compared to the vehicle treated CIA rats. Similarly, rats treated with cashew skin extract showed dose correlated improvement in the histopathology readings of ankle joints in relative to the vehicle treated CIA rats. In particular, animals treated with 200 mg/kg cashew skin extract showed 54.5%, 59.8%, 50.5% and 54.5% reductions in the severity of cartilage destruction, bone erosion, inflammation and GAG loss, respectively, when compared to the vehicle treated CIA rats. Among these reductions, the bone erosion and inflammation mitigations were statistically significant for the 200 mg/kg treatment group when compared to the vehicle treated CIA rats. Moderate reductions such as 35.7%, 51.2%, 45.3% and 35.7% were observed for cartilage destruction, bone erosion, inflammation, and GAG loss, respectively, for the CIA rats treated with 100 mg/kg cashew skin extract in relative to vehicle treated CIA rats. The bone erosion change was statistically significant for the 100 mg/kg treated rats in relative to CIA rats treated with vehicle. Animals treated with the 50 mg/kg cashew skin extract or the GC group showed very similar pattern in all categories evaluated in the histopathology, which were very comparable to the vehicle treated CIA rats with minimal to no activity in mitigating symptoms associated with CIA.

Summary of Efficacy of Anacardium occidentale L. Extract in Collagen-Induced Rat Paw Arthritis Induction—

Collagen induced arthritis (CIA) in rats was developed and utilized to evaluate efficacy of orally administered cashew testa extract for three weeks post disease induction. The study report includes seven groups of rats, with nine (9) rats per group. The rats in three of the groups were orally treated with cashew testa extract at three different dosages—a low dose of 50 mg/kg, a mid-dose of 100 mg/kg, and a high-dose of 200 mg/kg. The effectiveness of the cashew testa extract groups was compared against a group treated with the immunosuppressive drug Methotrexate dosed at 0.5 mg/kg, and a group treated with Glucosamine and chondroitin (150G+120C mg/kg) daily for three weeks. The normal control rats and CIA rats were treated with the carrier vehicle 0.5% Carboxymethyl cellulose only. During the in-life period, arthritis severity index, paw thickness, ankle diameter and pain sensitivity were monitored. Urine and serum were collected at the end of the study for biomarker analysis. At necropsy, the ankle joint from each rat was collected for histopathology analysis. Urinary cartilage degradation marker (CTX-II), proinflammatory cytokines (TNF-α, IL-1β, and IL-6), cartilage synthesis marker (PIIANP) and matrix degrading proteases (MMP13) were measured to determine the efficacy of each treatment.

Induction of disease model was confirmed by the progressively increased arthritis severity index and, later, by accompanied urinary and serum arthritis related biomarkers as well as histopathology findings. The rats showed various degrees of responses to the treatments. Cashew testa extract showed dose correlated and measurable efficacy with significant impact at higher dosages in mitigating the symptoms of arthritis. When the overall data for arthritis severity, paw thickness, ankle diameter and pain sensitivity were compared, CIA rats treated with mid- and high doses of cashew skin extract showed statistically significant reduction in the prime signs of arthritis. Rats in the GC and 50 mg/kg cashew skin extract group showed minimal efficacy in this study.

Data from the biomarkers were in accordance with in-life observations. Statistically significant reduction (53.4% inhibition vs. vehicle; P=0.04) in was observed for CIA rats treated with 200 mg/kg cashew testa extract when data were normalized with protein. Similarly, statistically significant reductions in serum IL-1 (200 mg/kg cashew testa extract, 53.1% reduction, P=0.01) and TNF-α (100 mg/kg cashew testa extract) levels were observed for rats treated with cashew testa extract when compared to the vehicle treated group. While the low dose cashew testa extract was infective in suppressing IL-1β and TNF-α, the GC treated rats showed statistically significant decrease in TNF-α. The cashew testa extract (100 mg/kg and 200 mg/kg) and GC treated group achieved the level of significance for the increases of anabolic marker when compared to the vehicle treated group.

Furthermore, the histopathology data were well aligned with the severity score of arthritis. While the vehicle-treated rats experienced severe synovitis, marked cartilage degeneration, diffused necrosis of bone and cartilage, synovial hyperplasia, pannus formation, bone erosion, and loss of architectural structure, CIA rats treated with cashew testa extract and methotrexate had relatively moderate morphological alternations in matrix integrity, and reduced articular bone damage. Cashew testa extract (200 mg/kg) treated rats showed statistically significant reductions in inflammation and bone erosion from the modified Mankin score analysis of histopathology data. Rats treated with GC or 50 mg/kg of cashew testa extract resulted in minimal microscopic improvement for articular structure damage.

Accordingly, based on data from the in-life measurements (arthritis severity, paw thickness, ankle diameter and pain sensitivity), urinary CTX-II, serum IL-1β and TNF-α, and histopathology analysis, cashew testa extract administered orally at 100 mg/kg or 200 mg/kg performed significantly superior to the GC treated group. Treatment of rats with GC produced statistically significant changes in the anabolic (PIIANP) markers and TNF-α. The collective data support the potential use of cashes testa extract for support joint structure and function.

The above description discloses several methods and materials of the present invention. This invention is susceptible to modifications in the methods and materials, as well as alterations in the fabrication methods and equipment. Such modifications will become apparent to those skilled in the art from consideration of this disclosure or practice of the invention disclosed herein. Further, unless defined otherwise, all technical and scientific terms used herein have the same meaning as commonly understood to one of ordinary skill in the art to which this invention belongs. Consequently, it is not intended that this invention be limited to the specific embodiments disclosed herein, but that it covers all modifications and alternatives coming within the true scope and spirit of the invention as embodied in the attached claims.

We claim:

1. A method for reducing joint stiffness and discomfort in a mammal in need thereof comprising:
administering a therapeutically effective amount of a composition comprising a botanical extract of the testa of Anacardium occidentale L, wherein the botanical extract is enriched for total catechin content, and
wherein the composition provides at least a 55.8% percent reduction in edema in a mammal with collagen-induced arthritis compared to that in a mammal with collagen-induced arthritis without the composition.

2. The method according to claim 1, wherein the botanical extract is standardized to a total catechin content of at least about 15.00% by weight, based on total weight of the extract.

3. The method according to claim 1, wherein the botanical extract is further enriched for total polyphenol content.

4. The method according to claim 1, wherein the method ameliorates cyclooxygenase and 5-lipoxygenase mediated inflammation in the mammal having joint stiffness and discomfort.

5. A method for improving cartilage rebuild or renewal function in a mammal in need thereof comprising:
administering a therapeutically effective amount of a composition comprising a botanical extract of the testa of Anacardium occidentale L, wherein the botanical extract is enriched for total catechin content, and
wherein the composition inhibits cartilage degradation in an amount of at least about 21.3% in a mammal with collagen-induced arthritis relative to a mammal with collagen-induced arthritis without the composition.

6. The method according to claim 5, wherein the botanical extract is standardized to a total catechin content of at least about 15.00% by weight, based on total weight of the extract.

7. The method according to claim 5, wherein the botanical extract is further enriched for total polyphenol content.

8. The method according to claim 5, wherein the method ameliorates cyclooxygenase and 5-lipoxygenase mediated inflammation in the mammal in need of cartilage rebuild or renewal function.
